(19) United States
(12) Patent Application Publication
(10) Pub. No.: US 2008/0031887 A1
(43) Pub. Date: Feb. 7, 2008

(54) Conjugates for inducing targeted immune responses and methods of making and using same

(76) Inventor: Joseph Lustgarten, Scottsdale, AZ (US)

Correspondence Address: DUNLAP CODDING & ROGERS, P.C., PO BOX 16370, OKLAHOMA CITY, OK, 73113, US

(21) Appl. No.: 11/823,921

(22) Filed: Jun. 29, 2007

Related U.S. Application Data
(60) Provisional application No. 60/818,023, filed on Jun. 30, 2006.
(60) Provisional application No. 60/818,629, filed on Jul. 5, 2006.

(51) Int. Cl. A61K 39/395; A61K 39/39; A61P 31/00; A61P 35/00; A61P 37/00

(52) U.S. Cl. 424/179.1; 424/178.1; 424/193.1

(57) ABSTRACT

Disclosed herein are methods of producing conjugates that include Toll-like receptor (TLR) ligands and targeting molecules that are effective in inducing an immune response. Also disclosed are methods of using such conjugate to treat a disease, such as cancer, infectious diseases and autoimmune diseases.

### CROSS-REFERENCE TO RELATED APPLICATIONS

This application claims benefit under 35 U.S.C. 119(e) of provisional applications U.S. Ser. No. 60/818,023, filed Jun. 30, 2006; and U.S. Ser. No. 60/818,629, filed Jul. 5, 2006. The entire contents of each of the above-referenced patent applications are hereby expressly incorporated herein by reference.

### STATEMENT REGARDING FEDERALLY SPONSORED RESEARCH OR DEVELOPMENT

Not Applicable.

### BACKGROUND OF THE INVENTION

1. Field of the Invention

The present invention relates generally to a methodology of producing conjugates effective in inducing an immune response, as well as methods of using same, and more particularly, but not by way of limitation, to a methodology of producing conjugates comprising Toll-like receptor (TLR) ligands and targeting molecules that are effective in inducing an immune response, as well as methods of using same to treat a disease such as but not limited to cancer, infectious diseases and autoimmune diseases.

2. Description of the Background Art

Immunotherapeutic strategies designed to induce a cellular immune response have recently received much attention as promising approaches for the treatment of many types of cancers. The discovery of tumor associated antigens (TAA) has been an important breakthrough in tumor immunology, because it is possible to devise immunotherapeutic approaches to promote T cell responses against such antigens and induce a protective immunity against neoplastic malignancies. The TAA can be classified into four families based on their expression and recognition patterns of T cells. The first family is known as cancer-testes antigens (CTAs). These proteins are normally expressed only in testes, but are aberrantly expressed in melanoma, bladder, colon, lung, prostate and other cancers. The NY-ESO-1 and the MAGE families are proteins that characterize this group. The second family includes the differentiation antigens, such as but not limited to, the melanocyte lineage. These antigens show a lineage specific expression in tumors (melanomas) and are also expressed in normal cells of the same origin. The tyrosinase or gp100 antigens are examples of this group. The third family of antigens includes viral-based proteins. These are cancers induced by viruses, for example but not by way of limitation, the human papillomavirus (HPV 16) that induces cervical cancer. Antigens such as E6 and E7 from HPV 16 can be recognized by T cells and used as targets for tumor protection. The fourth family includes “self-antigens” that are over expressed in the tumor when compared to the level of expression in normal cells. The Her-2/neu and p53 antigens are examples of this family. Significant progress has been made in the past decade in the identification of tumor-associated antigens. More than 170 antigenic peptides derived from 60 human tumor antigens are expressed and are recognizable by cells in the available T cell repertoire. Many candidate peptides have been used in clinical trials in efforts to develop therapeutic cancer vaccines, but most of these are poor immunogens and have failed to elicit measurable immune responses in the majority of the patients immunized.

The Her-2/neu antigen has been used herein as a tumor model. The Her-2/neu protein is a transmembrane glycoprotein with tyrosine kinase activity whose structure is similar to that of the epidermal growth factor receptor. The Her-2/neu protein is a component of a four member family of closely related growth factor receptors including EGFR or Her-1, Her-3 and Her-4. Amplification of the Her-2/neu gene was reported in various types of cancers, including ovarian, gastric, colon, prostate and especially breast. The Her-family of receptors plays a role in the process of growth signal transduction across the cell membrane. Consequently, overexpression of one or more of these proteins contributes to uncontrolled growth signal transduction and, therefore, cellular transformation. Overexpression of Her-2/neu is associated with metastatic disease, poor prognosis and low survival. Additionally, tumors overexpressing Her-2/neu show lower responsiveness to adjuvant therapy that includes cyclophosphamide, methotrexate and 5′ fluorouracil. Furthermore, the Her-2/neu protein seems to synergize with the multi drug resistant protein, p170mdr-1, rendering breast cancer more resistant to taxol. Studies with gene knockouts have demonstrated that target deletion of Her-2/neu is embryonically lethal, indicating that the Her-2/neu gene is involved in the early stages of development.

T cell immunity is a critical component of the immune response to a growing tumor. Although the identification of TAA encoding mutated cellular genes serves as targets for T cell immunity, the majority of the currently defined TAA are often overexpressed products of normal cellular genes. Therefore, in practice these overexpressed proteins pose a significant challenge to the design of effective T cell immunotherapies due to considerations of self-tolerance. Based on transgenic mouse models, it is now clear that tolerance is capable of deleting reactive high avidity T cells against the transgene (self), thereby leading to self tolerance. However, T cell elimination through tolerance is not absolute, and self-specific T cells can be isolated from tolerant hosts. A characteristic of these self-specific T cells is that the majority of them have low avidity for the antigen. The significance of understanding the mechanism responsible for the persistence of low avidity T cells relates not only to an understanding of autoimmunity, but also to the potential for targeting such cells against self-tumor antigens for tumor destruction. Therefore, a central question is whether the available repertoire of T cells specific for up-regulated tumor-self antigens is sufficient in number or avidity to mount an effective antitumor response. This fundamental question has been addressed by the present invention using an experimental model in which the Her-2/neu protooncogene is expressed in the mammary tissue under the control of the MMTV promoter (FVB-neu transgenic mice) (Muller et al., 1988; and Guy et al., 1992). Additionally, the clinical progression and pathogenesis of the disease in these mice closely resembles what is seen in human patients with breast cancer. Therefore, the neu mouse is a clinically relevant animal tumor model that can be used: 1) to define the nature of the responsiveness to self-tumor antigens; 2) to analyze the requirements for initiating and sustaining antitumor responses in tolerant hosts; and 3) to evaluate strategies for overcoming or circumventing tolerance to self tumor antigens that can be effectively used as targets for immunotherapy.

As has been demonstrated with other transgenic models, the expression of a protein (i.e., hemagglutinin, HA) as a self-protein induces tolerance to the protein (Lo et al., 1992). Thus, it was desired to evaluate the immune responses to neu antigens in neu mice. However, since there are no known H2q (haplotype of FVB mice) epitopes for the neu protein and in order to be able to evaluate peptide specific immune responses in neu mice, the neu mice were crossed with the A2.1/Kb transgenic mouse (Vitiello et al., 1991) so that A2.1-Her-2/neu responses could be evaluated against the p369-377 and p773-782 peptides that have previously been identified by the inventor (Lustgarten et al., 1997). For the first time, the F1 animals (A2×FVB-neu) allowed the study of peptide specific responses in neu mice. As expected, T cells obtained from A2×FVB-neu mice were less efficient in recognizing target cells loaded with the peptides than compared to T cells derived from A2×FVB mice (A2.1/Kb transgenic mice crossed with FVB wild type mice). These results showed that the A2×FVB-neu mice contained only a low avidity repertoire to neu antigens. In addition, these results are in agreement with the findings of other laboratories showing that neu mice are tolerant to neu antigens (Reilly et al., 2000; Reilly et al., 2001; and Kurt et al., 2000). Next, it was evaluated whether the residual repertoire for neu antigens was effective in inducing an antitumor response. The inventor has previously demonstrated that multiple immunizations with dendritic cells (DCs) pulsed with the neu-antigens in combination with anti-OX40 or anti-4-1-BB monoclonal antibody (mAb) induced a stronger antitumor response than compared to animals immunized with DC-pulsed with neu antigens (Cuadros et al., 2003; Lustgarten et al., 2004; and Cuadros et al., 2005). Although the these results demonstrated the ability to improve the antitumor responses in neu mice, these results indicated that the immune responses induced after DC-vaccination were not sufficient for controlling the tumor growth in Her-2/neu tolerant mice. This raises the question of which conditions should be optimized for maximizing the antitumor response in tolerant hosts.

The immune system can be divided into innate and adaptive components. The innate immune response is the first line of defense against infectious diseases, while the adaptive immune responses represent specific resistance, weak at first but developing into long-term memory responses. More importantly, adaptive responses are initiated when T and B cells recognize foreign molecules expressed on antigen presenting cells (APC). The major difference between the innate and adaptive immune systems lies in the mechanism of recognition of antigens. In the adaptive immune response, T and B cell responses recognize the antigen through the T and B cell receptors, respectively, which have the capacity to recognize almost any antigen structure. Additionally, each T or B cell expresses a unique receptor that can bind any antigen regardless its origin. The innate response is largely mediated by white blood cells such as neutrophils, monocytes, macrophages (MΦ) and dendritic cells (DCs). In contrast to the adaptive immune response, the innate immune response relies on the recognition of the antigen by receptors that recognize specific structures found exclusively in microbial pathogens termed pathogen-associated molecular patterns (PAMPs) (Barton et al., 2002). The recognition of PAMPs by the innate immune system can regulate the induction of adaptive immune responses (Huang et al., 2001). For example, DCs respond to some microbial product by taking up the antigen. Concurrently, DCs synthesize a wide variety of inflammatory mediators and cytokines amplifying the immune response and, additionally, DCs can process and present antigens resulting in the activation of T and B cell responses and the establishment of protective immunity. Therefore, a number of microbial products are thought to function as effective adjuvants due to effects on APCs, which in turn, can influence the activation of an adaptive immune response.

More than a decade ago, Janeway postulated that regulation of PAMPs recognition must be controlled by receptors with a specificity for microbial products, thereby linking innate recognition of non-self with the induction of adaptive immunity (Janeway, 1989). Recent studies have demonstrated that recognition of PAMPs by APCs is mediated by a Toll-like receptor (TLRs) family (Means et al., 2000; and Kaisho et al., 2002). There are currently 10 known TLR family members capable of sensing bacterial wall components, such as LPS (TLR-2/4), lipoteichoic acids (TLR-2/4), CpG-DNA (TLR-9), flagellin (TLR-5), as well as other microbial products (Takeda et al., 2003). A wide variety of TLRs are expressed in immature or mature DCs, MΦ and monocytes; and these receptors control the activation of those APCs (Aderem et al., 2000). Recognition of PAMPs by TLRs initiates a signaling pathway that leads to activation of NF-kB transcription factors and members of the MAP kinase family (Means et al., 2000). All TLRs share a common intracellular domain that is similar to the IL-1 receptors. The signal is mediated through the adaptor protein MyD88 (Takeuchi et al., 2002). The TLRs signaling triggers maturation and activation of APCs that includes upregulation of MHC and co-stimulatory molecules, and secretion of pro-inflammatory cytokines and chemokines (Gewirtz et al., 2001). This maturation of APCs significantly increases their ability to prime naive T cells. In this way, TLRs link the recognition of pathogens with induction of adaptive immune responses.

Bacterial DNA or synthetic oligonucleotides containing unmethylated CpG motifs (CpG-ODN) stimulate vertebrate immune cells both in vitro as well as in vivo (Krieg, 2002). In contrast, mammalian DNA has a very low frequency of CpG dinucleotides, and those are mostly methylated; therefore, mammalian DNA does not have the same immunostimulating activities (Yamamoto et al., 1992). The mammalian immune system has apparently evolved so that it can recognize CpG-ODN molecules as an early sign of infection, as part of initiating an immediate and powerful immune response against invading pathogens or other foreign organisms. CpG-ODN induces both B cells and DC to express increased levels of co-stimulatory molecules, and to secrete Th1 promoting chemokines and cytokines. Additionally, the DCs produce high levels of type I IFN (58), and the B cells secrete antibodies (Hartmann et al., 2000). The direct effects of CpG-ODN lead to secondary effects including the rapid activation of DC, macrophage and NK cell activity (Balas et al., 1996). Activation of B cells and DCs stimulates naïve T cells differentiation into Th1 cells and effector CTLs (Liang et al., 1996; and Hartmann et al., 2000). The pattern and kinetics of cytokine production are also affected by CpG-ODNs (Jakob et al., 1998; Ueno et al., 2000; and Yamamoto et al., 2003). Injections of CpG-ODN modulate the immune response in different ways. For example, intra muscular injections of CpG-ODN induced the expression of gene coding for chemokines and MHC class II molecules on myocytes (Stan et al., 2001), while in-vitro studies also demonstrated that macrophages exposed to CpG-ODN up-regulated expression of mRNA encoding the chemokines MIP-1α, MIP-1b, MIP-2, RANTES, MCP-1 and IP-10 (Takeshita et al., 2000). In human studies, CpG-ODN is also reported to be a more potent agent than GM-CSF for dendritic cell activation, maturation, and their functional ability to promote a Th1-like T cell response (Jakob et al, 1998). These data led investigators to postulate that CpG-ODNs could act as useful adjuvants for the development of vaccines or vaccination strategies. The utility of CpG-ODN as a vaccine adjuvant has been confirmed in studies using a wide range of antigens, including protein or peptide antigens, live or killed viruses, DC vaccines or fusion peptides (68-70). Many reports have demonstrated the utility of CpG-ODNs as therapeutic agents in cancer immunotherapy (Davila, et al., 2002; Heit et al., 2005; and Davila et al., 2003). For example, Davila and Celis (2000) showed that tumor protein vaccination combined with CpG-ODN resulted in increased cytotoxic T cell activity, and in delayed tumor growth, thereby extending survival in mice bearing melanoma tumors. Additionally, CpG-ODN therapy is also reported to induce the regression of mouse neuroblastoma following peritumoral injection (Heckelsmiller et al., 2002). Furthermore, it has been reported that CpG-ODN therapy induced the regression of intracranial gliomas following intratumoral injection (Carpentier et al., 2000). However, the anti-tumor effect of CpG-ODN in tolerant hosts has not previously been considered.

As shown herein below, the presently claimed and disclosed invention demonstrates that intratumoral (i.t.) injections of CpG-ODN induced the complete rejection of tumors in Her-2/neu mice. In contrast, i.t. injection of control-ODN or systemic injections of CpG-ODN did not affect the tumor growth. Currently there are no studies evaluating the anti-tumor effect of CpG-ODN in tolerant hosts, and for the first time these results have shown that i.t. injections of CpG-ODN overcome tolerance. The presently disclosed and claimed invention thus demonstrates that manipulation of the tumor microenvironment by direct tumor injection of CpG-ODN results in a stronger antitumor response, thereby controlling the tumor growth in Her-2/neu tumor bearing mice.

These results indicate that intratumoral CpG-ODN treatments induce strong antitumor responses and are potentially a good strategy for overcoming tolerance. However, the major drawback of this strategy is that not all tumors will be physically available for intratumoral injections, and it will be difficult to target metastatic lesions. Thus, the scope of the presently disclosed and claimed invention includes the use of CpG-ODN-targeted immunotherapy as an efficient strategy for the complete elimination of tumors. In order to target the CpG-ODN at the tumor site anywhere in the body, an antibody-CpG-ODN conjugate was generated. An anti-Her-2/neu mAb was chemically conjugated with CpG-ODN. The presently disclosed and claimed invention demonstrates that the anti-neu-CpG-ODN conjugate retains its abilities to (1) bind to Her-2/neu+ tumors and (2) activate and induce the maturation of DCs, thus demonstrating that the anti-neu-CpG-ODN conjugate is a functional molecule. The presently disclosed and claimed invention is thus directed to the use of the anti-neu-CpG-ODN conjugated molecule as a novel strategy for controlling primary and metastatic tumors, as demonstrated in the Her-2/neu mouse model system. The presently disclosed and claimed invention also encompasses a new strategy for the treatment of localized and disseminated tumors and for targeting other tumor antigens with different antibodies.

### BRIEF DESCRIPTION OF THE DRAWINGS

FIG. 1 contains graphic representations illustrating that intratumoral injections of CpG-ODN induce rejection of tumors in Balb/c, BALB-neuT and A2×neu mice. Balb/c and BALB-neuT were implanted s.c. with 10⁶ TUBO cells, and A2×neu mice were implanted s.c. with 10⁶ N202.A2 cells on day zero. On day 10, animals started treatment with i.t. injections of TLR-ligands three times a week (20 μg/injection) for three weeks.

FIG. 2 contains a graphic representation illustrating that anti-tumor responses induced by CpG-ODN are dependent on CD4⁺, CD8⁺ T cells and NK cells.

FIGS. 3A and 3B contain graphic representations illustrating the results obtained when A2×neu and A2×FVB mice were immunized with DCs pulsed with the p369-377 and p773-783 peptides, and restimulated in vitro. (A) Staining with A2.1-p369-377- PE tetramer. (B) Staining with A2.1-p773-782- PE tetramer. FIGS. 1C and 1D contain graphic representations illustrating lytic activity of spleen cells from p369-377 (C) and p773-782 (D) peptide immunized animals. Stimulated spleen cells were assayed at an E:T ratio of 10:1 for cytotoxicity against T2-A2/Kb target cells pulsed with their respective peptides.

FIG. 4 contains graphic representations illustrating lysis of N202.A2 cells by p369-377 and p773-782 CTLs. The p369-377 and p773-782 CTLs from A2×FVB (A) and A2×neu (B) mice were assayed for the cytotoxic activity of ⁵¹Cr-labeled N202.A2 and N202.

FIG. 5 contains graphic representations illustrating the results obtained when A2×neu mice were inoculated s.c. on day zero with 10⁶ N202.A2 cells. Animals were immunized three times (on days 7, 17 and 27) with s.c. injections of 10⁶ DCs pulsed with the p773 peptide. (A) anti-OX40 or (B) anti-4-1 BB (100 μg/injection) was administered two days after each immunization.

FIG. 6 contains a graphic representation illustrating that intratumoral injections of CpG-ODN induce rejection of tumors in neu mice. neu mice were inoculated s.c. on day 0 with 10⁶ N202 cells and treated with CpG-ODN or Control-ODN.

FIG. 7 illustrates two reaction pathways for preparation of antibody/CpG oligo conjugates. FIG. 7A: synthetic pathway used to prepare a non-cleavable antibody/CpG oligo conjugate; FIG. 7B: synthetic pathway used to prepare a disulfide cleavable antibody/CpG oligo conjugate. FIG. 7C: schematic representation of the anti-neu-CpG-ODN conjugated molecule produced in accordance with the present invention.

FIG. 8 contains a graphic representation illustrating binding of anti-neu-CpG-ODN to N202 cells. The thin line represents the control, the thick line represents anti-neu, and the broken line represents anti-neu-CpG-ODN.

FIG. 9 contains a graphic representation illustrating binding of anti-neu-CpG-ODN to TUBO cells. Anti-neu and anti-neu-CpG-ODN were diluted 1/10 (thick line), 1/100 (broken line) and 1/1000 (dash line). The thin line represents the control.

FIG. 10 contains a graphic representation illustrating that CpG-ODN and anti-neu-CpG-ODN induce the expression of activator markers on DCs.

FIG. 11 contains a graphic representation illustrating that DC stimulation with CpG-ODN or anti-neu-CpG-ODN induced the secretion of TNF-α.

FIG. 12 contains graphic representations illustrating that Anti-neu-CpG-ODN bound onto N202 cells induce the activation of DCs. (A) Expression of Class I and (B) B7.1 molecules. Thin line: DC incubated with N202; Broken line: DC incubated with N202 plus anti-neu mAb; Thick line: DC incubated with N202 plus anti-neu-CpG-ODN. (B) TNF-a secretion.

FIG. 13 contains graphic representations illustrating the antitumor effect of anti-neu-CpG-ODN. Balb/c and BALB/neuT were implanted s.c. with 10⁶ TUBO cells on day zero. On day 10, animals started treatment with i.t. or i.v. injections of CpG-ODN, anti-neu-CpG-ODN, anti-neu or their combinations twice a week (50 μg/injection) for three weeks.

FIG. 14 contains a graphic representation illustrating the antitumor effect of anti-neu-CpG-ODN containing a cleavable or non-cleavable bond. Balb/c mice were implanted s.c. with 10⁶ TUBO cells on day zero. Starting on day 10, animals were injected intratumorally with the different anti-neu-CpG-ODN molecules, three times a week (30 μg/injection) for three weeks. Survival of animals was evaluated.

### DETAILED DESCRIPTION OF THE INVENTION

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The terms “oligonucleotide,” “polynucleotide,” and “nucleic acid molecule”, used interchangeably herein, refer to polymeric forms of nucleotides of any length, wherein the nucleotides may be ribonucleotides, deoxyribonucleotides or a modified form of either type of nucleotide. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites, and/or phosphorothioates, and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. Other examples of oligonucleotide linkages that may be utilized in accordance with the present invention include phosphorodithioate, phosphoroselenoate, phosphorodiselenoate, phosphoroanilothioate, phosphoraniladate, and the like. See e.g., LaPlanche et al. Nucl. Acids Res. 14:9081 (1986); Stec et al: J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. The polynucleotide may comprise one or more L-nucleosides. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be modified to comprise N3′-P5′ (NP) phosphoramidate, morpholino phosphorociamidate (MF), locked nucleic acid (LNA), 2′-O-methoxyethyl (MOE), or 2′-fluoro, arabino-nucleic acid (FANA), which can enhance the resistance of the polynucleotide to nuclease degradation (see, e.g., Faria et al. (2001) Nature Biotechnol. 19:40-44; Toulme (2001) Nature Biotechnol. 19:17-18). A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Immunomodulatory nucleic acid molecules can be provided in various formulations, e.g., in association with liposomes, microencapsulated, etc., as described in more detail herein.

The term “polynucleotide” as referred to herein means a polymeric form of nucleotides of at least 10 bases in length. The term “oligonucleotide” as used herein refers to a polynucleotide subset generally comprising a length of 200 bases or fewer. Oligonucleotides are usually single stranded, although oligonucleotides may be double stranded. Oligonucleotides of the invention can be either sense or antisense oligonucleotides.

The terms “polypeptide,” “peptide,” and “protein”, used interchangeably herein, refer to a polymeric form of amino acids of any length, which can include coded and non-coded amino acids, chemically or biochemically modified or derivatized amino acids, and polypeptides having modified peptide backbones. The term includes polypeptide chains modified or derivatized in any manner, including, but not limited to, glycosylation, formylation, cyclization, acetylation, phosphorylation, and the like. The term includes naturally-occurring peptides, synthetic peptides, and peptides comprising one or more amino acid analogs. The term includes fusion proteins, including, but not limited to, fusion proteins with a heterologous amino acid sequence, fusions with heterologous and homologous leader sequences, with or without N-terminal methionine residues; immunologically tagged proteins; and the like.

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, preferably 75% free, and most preferably 90% free from other components with which it is naturally associated.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or 1) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-,α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

Similarly, unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules or fragments thereof are contemplated as being encompassed by the present invention, providing that the variations in the amino acid sequence maintain at least 75%, and in some embodiments at least 80%, 90%, 95%, and 99%, sequence identity. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

In one embodiment, amino acid substitutions are those which: (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various mutations of a sequence other than the naturally-occurring peptide sequence. For example, single or multiple amino acid substitutions (such as conservative amino acid substitutions) may be made in the naturally-occurring sequence (such as in the portion of the polypeptide outside the domain(s) forming intermolecular contacts). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure©. Branden and J. Tooze, eds., Garland Publishing, New York, N.Y. (1991)); and Thornton et at. Nature 354:105 (1991), which are each expressly incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, such as at least 14 amino acids long, or at least 20 amino acids long, or at least 50 amino acids long, or at least 70 amino acids long.

“Antibody” or “antibody peptide(s)” as used herein refer to an intact antibody or immunoglobulin molecule, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)2 and Fv) so long as they exhibit the desired biological activity. Antibodies (Abs) and immunoglobulins (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond. While the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985).

An “isolated” antibody is one that has been identified and separated and/or recovered from a component of the environment in which it was produced. Contaminant components of its production environment are materials that would interfere with diagnostic or therapeutic uses for the antibody, and may include enzymes, hormones, and other proteinaceous or nonproteinaceous solutes. In preferred embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, and more preferably at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

The term “antibody mutant” refers to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95%, amino acid sequence identity.

The term “antibody fragment” refers to a portion of a full-length antibody, generally the heavy chain or constant domain. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)2 fragment that has two antigen binding fragments which are capable of cross-linking antigen, and a residual other fragment (which is termed pFc′).

An “Fv” fragment is the minimum antibody fragment that contains a complete antigen recognition and binding site. This region consists of a dimer of one heavy and one light chain variable domain in a tight, non-covalent association (VH-VL dimer). It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The “Fab” fragment [also designated as “F(ab)”] also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)2 pepsin digestion product. Additional chemical couplings of antibody fragments are known to those of ordinary skill in the art.

The “Fc” fragment is a crystallizable, non-antigen-binding fragment of an immunoglobulin that consists of the carboxyl-terminal portions of both heavy chains, which possess binding sites for Fc receptors and the C1q component of complement. The Fc fragment forms the stem of the “Y” structure of an immunoglobulin molecule, and is composed of two heavy chains that each contribute two to three constant domains (depending on the class of the antibody). By binding to Fc receptors and the C1q component of complement, the Fc fragment mediates different physiological effects of antibodies (such as but not limited to, opsonization, cell lysis, mast cell, basophil and eosinophil degranulation and other processes).

Fc receptors are cell-surface receptors specific for the Fc portion of certain classes of immunoglobulin. Fc receptors are present on cell types such as lymphocytes, mast cells, macrophages, and other accessory cells.

The light chains of antibodies (immunoglobulin) from any vertebrate species can be assigned to one of two clearly distinct types, called kappa (.kappa.) and lambda (.lambda.), based on the amino sequences of their constant domain.

Depending on the amino acid sequences of the constant domain of their heavy chains, “immunoglobulins” can be assigned to different classes. There are at least five (5) major classes of immunoglobulins: IgA, IgD, IgE, IgG and IgM, and several of these may be further divided into subclasses (isotypes), e.g., IgG-1, IgG-2, IgG-3 and IgG4; IgA-1 and IgA-2. The heavy chains constant domains that correspond to the different classes of immunoglobulins are called α, Δ, ε, γ and μ, and correspond to IgA, IgD, IgE, IgG, and IgM, respectively. The subunit structures and three-dimensional configurations of different classes of immunoglobulins are well known.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, such as more than about 85%, 90%, 95%, and 99%. In one embodiment, the object species is purified to essential homogeneity (contaminant species cannot be detected in the composition by conventional detection methods) wherein the composition consists essentially of a single macromolecular species.

The term “Toll-like receptor” or “TLR” as used herein will be understood to refer to type I transmembrane proteins that recognize pathogens and activate immune cell responses as a key part of the innate immune system. In vertebrates, they can help activate the adaptive immune system, linking innate and acquired immune responses. TLR are pattern recognition receptors (PRRs), binding to pathogen-associated molecular patterns (PAMPs), small molecular sequences consistently found on pathogens. TLRs function as a dimer. Though most TLRs appear to function as homodimers, TLR2 forms heterodimers with TLR1 or TLR6, each dimer having a different ligand specificity. TLRs may also depend on other co-receptors for full ligand sensitivity, such as in the case of TLR4's recognition of LPS, which requires MD-2. CD14 and LPS Binding Protein (LBP) are known to facilitate the presentation of LPS to MD-2. The function of TLRs in all organisms appears to be similar enough to use a single model of action. Each Toll-like receptor forms either a homodimer or heterodimer in the recognition of a specific or set of specific molecular determinants present on microorganisms. Because the specificity of Toll-like receptors (and other innate immune receptors) cannot be changed, these receptors must recognize patterns that are constantly present on threats, not subject to mutation, and highly specific to threats (i.e. not normally found in the host where the TLR is present.) Patterns that meet this requirement are usually critical to the pathogen's function and cannot be eliminated or changed through mutation; they are said to be evolutionarily conserved. Well-conserved features in pathogens include bacterial cell-surface lipopolysaccharides (LPS), lipoproteins, lipopeptides and lipoarabinomannan; proteins such as flagellin from bacterial flagella; double-stranded RNA of viruses or the unmethylated CpG islands of bacterial and viral DNA; and certain other RNA and DNA. The terms “Toll-like receptor” and “TLR” as used herein will further be understood to include other pattern recognition receptors that recognize pathogen-associated molecular patterns (PAMPS).

The terms “Toll-like Receptor Ligand”, “TLR ligand”, “Pathogen-Associated Molecular Pattern” and “PAMP” are used herein interchangeably and will be understood to refer to small molecular sequences consistently found on pathogens that are recognized by toll-like receptors and other pattern recognition receptors (PRRs).

Table I provides a summary of known TLR's and their respective ligands.

TABLE I
Summary of Known Mammalian Toll-Like Receptors

| Receptor | Ligand PAMP(s) |
|---|---|
| TLR 1 | Triacyl lipoproteins |
| TLR 2 | Lipoproteins; gram positive peptidoglycan; lipoteichoic |
| | acids; fungi; viral glycoproteins |
| TLR 3 | Double-stranded RNA (as found in certain viruses); |
| | poly I:C |
| TLR 4 | Lipopolysaccharide; viral glycoproteins |
| TLR 5 | Flagellin |
| TLR 6 | Diacyl lipoproteins |
| TLR 7 | Small synthetic compounds; single-stranded RNA |
| TLR 8 | Small synthetic compounds; single-stranded RNA |
| TLR 9 | Unmethylated CpG DNA |
| TLR 10 | Unknown |
| TLR 11 | Unknown, but present in uropathogenic bacteria |

The term “CpG” as used herein will be understood to refer to oligonucleotides containing a region where a cytosine nucleotide occurs next to a guanine nucleotide in the linear sequence of bases along its length. “CpG” stands for cytosine and guanine separated by a phosphate, which links the two nucleosides together in DNA.

The term “ODN” as used herein is defined as oligodeoxynucleotide.

The terms “immunomodulatory nucleic acid molecule,” “ISS,” “ISS-PN,” and “ISS-ODN,” are used interchangeably herein, and refer to a polynucleotide that comprises at least one immunomodulatory nucleic acid moiety. The term “immunomodulatory,” as used herein in reference to a nucleic acid molecule, refers to the ability of a nucleic acid molecule to modulate an immune response in a vertebrate host.

The term “targeting molecule” as used herein will be understood to refer to any protein, peptide or fragment thereof having the ability to bind to a receptor present on a surface of a particular cell.

The terms “cleavable linkage” and “cleavable bond” as used herein refer to a chemical or covalent bond occurring in a molecule, wherein the chemical or covalent bond can be broken, resulting in two smaller molecules.

The term “TUBO cells” as used herein will be understood to refer to a cloned cell line generated from a spontaneous mammary gland tumor from a BALB-neuT mouse and highly expresses HER-2 protein on the cell membrane.

The terms “treatment” or “treating” as used herein refer to any therapeutic intervention in a subject, usually a mammalian subject, generally a human subject, including but not limited to: (i) prevention, that is, causing the clinical symptoms not to develop, e.g., preventing infection, tumor growth, and/or preventing progression to a harmful state; (ii) inhibition, that is, arresting the development or further development of clinical symptoms, e.g., mitigating or completely inhibiting an active (ongoing) infection so that pathogen load is decreased to the degree that it is no longer harmful, which decrease can include complete elimination of an infectious dose of the pathogen from the subject, or arresting the development of tumor growth and/or metastasis as well as decreasing tumor size; and/or (iii) relief, that is, causing the regression of clinical symptoms, e.g., causing a relief of symptoms caused by an infection, a cancer, or an autoimmune disorder.

The term “effective amount” or “therapeutically effective amount” means a dosage sufficient to provide for treatment for the disease state being treated or to otherwise provide the desired effect (e.g., induction of an effective immune response). The precise dosage will vary according to a variety of factors such as subject-dependent variables (e.g., age, immune system health, etc.), the disease, and the treatment being effected. In the case of an intracellular pathogen infection, an “effective amount” is that amount necessary to substantially improve the likelihood of treating the infection, in particular that amount which improves the likelihood of successfully preventing infection or eliminating infection when it has occurred. In the case of a cancer, an “effective amount” is that amount necessary to substantially decrease the size of the tumor and prevent further spread of the cancer cells to other tissues.

The term “disorder” as used herein refers to any condition that would benefit from treatment with the conjugate of the present invention. This includes chronic and acute disorders or diseases including those pathological conditions that predispose the mammal to the disorder in question.

The terms “cancer” and “cancerous” as used herein refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hopatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

As used herein, the term “pathogen” or “intracellular pathogen” or “microbe” refers to any organism that exists within a host cell, either in the cytoplasm or within a vacuole, for at least part of its reproductive or life cycle. Intracellular pathogens include viruses, bacteria, protozoa, fungi, and intracellular parasites.

The term “Mammal” for purposes of treatment refers to any animal classified as a mammal, including human, domestic and farm animals, nonhuman primates, and zoo, sports, or pet animals, such as dogs, horses, cats, cows, etc. The term “patient” as used herein includes human and veterinary subjects.

The present invention relates to methodology of producing conjugates effective in inducing an immune response, as well as methods of using same, and more particularly, but not by way of limitation, to a methodology of producing conjugates comprising Toll-like receptor (TLR) ligands and targeting molecules, wherein the conjugates are effective in inducing an immune response, as well as methods of using same to treat a disease, such as, but not limited to, cancer, infectious diseases, and autoimmune diseases.

In one embodiment, the present invention provides a conjugate that includes at least one Toll-like receptor (TLR) ligand and a targeting molecule, conjugated together via a cleavable linkage. The targeting molecule is a peptide or protein that is a ligand for a receptor present on a surface of a desired cell, and the targeting molecule functions to target the conjugate to the desired cell.

In one embodiment, the at least one TLR ligand comprises at least one oligonucleotide containing at least one unmethylated CpG dinucleotide, and the targeting molecule comprises an immunologically active portion of an immunoglobulin heavy chain. The cleavable linkage between the oligonucleotide and the targeting molecule may be a cleavable disulfide linkage.

In another embodiment of the present invention, a conjugate is provided that includes at least one Toll-like receptor (TLR) ligand and a targeting molecule, conjugated together via a non-cleavable linkage. The targeting molecule is a peptide or protein that is a ligand for a receptor present on a surface of a desired cell, and the targeting molecule functions to target the conjugate to the desired cell. In one embodiment, the at least one TLR ligand comprises at least one oligonucleotide containing at least one unmethylated CpG dinucleotide, and the targeting molecule comprises an immunologically active portion of an immunoglobulin heavy chain.

The present invention is also related to a method of inducing a targeted inflammatory response. The method includes providing at least one of the conjugates described above, and administering an effective amount of the conjugate to a subject to induce an inflammatory response at a targeted location.

In one embodiment, the target location may be a tumor.

The present invention is also related to a method of treating a disease by administering to a patient, in need thereof, an effective amount of at least one of the conjugates described herein above. The patient may be suffering from a cancer, an infectious disease, or an autoimmune disease.

The presently claimed and disclosed invention also provides methods of stimulating a T cell response by administering an effective amount of the conjugates described herein.

The present invention is also related to a vaccine comprising at least one of the conjugates described herein above, as well as methods of making and using such vaccine.

The methods of the presently claimed and disclosed invention begin with the production of a conjugate. The conjugate comprises at least one Toll-like receptor (TLR) ligand and at least one targeting molecule conjugated together via a cleavable linkage. The targeting molecule is a peptide or protein that is a ligand for a receptor present on a surface of a desired cell, and the targeting molecule functions to target the conjugate to the desired cell.

The at least one TLR ligand may be any of the TLR ligands or PAMPs described herein above or known in the art, including but not limited to, lipoproteins, lipopolysaccharide, poly I:C, CpG-ODN, flagellin, gram positive peptidoglycan, lipoteichoic acids, fungi or viral glycoproteins, and the like.

In one embodiment, the at least one TLR ligand is an oligonucleotide containing at least one unmethylated CpG dinucleotide. CpG-ODNs are known in the art as immunostimulatory agents; however, the prior art only discloses local stimulation of immune responses using CpG, and prior to the present invention, methods of targeting CpG to a desired location were not known. Examples of CpG-ODNs that may be utilized in accordance with the present invention have been disclosed in U.S. Pat. No. 5,663,153, issued to Hutcherson et al. on Sep. 2, 1997; U.S. Pat. No. 6,194,388, issued to Krieg on Feb. 27, 2001; U.S. Pat. No. 5,856,462, issued Jan. 5, 1999 to Agrawal; U.S. Pat. No. 6,406,705, issued to Davis et al. on Jun. 18, 2002 (see, in particular, Table I thereof, which lists 98 different CpG-ODNs); U.S. Pat. No. 6,214,806, issued Apr. 10, 2001 to Schwartz et al.; U.S. Pat. No. 6,653,292, issued Nov. 25, 2003 to Krieg et al.; and U.S. Pat. No. 6,426,334, issued Jul. 30, 2002 to Agrawal et al. The contents of each of the above-referenced patents are hereby expressly incorporated herein by reference.

Any size CpG-ODNs may be utilized in accordance with the present invention. The only requirement of the CpG-ODN utilized in accordance with the present invention is that either the 5′ or 3′ end of the molecule be accessible for conjugation to the targeting molecule via a cleavable linkage.

However, it is to be understood that other molecules that can be conjugated with the targeting molecule and can function to activate an immune response may be utilized in accordance with the present invention. In another embodiment of the present invention, the conjugate includes any other type of immunomodulatory nucleic acid molecule or ISS described herein or known in the art.

The targeting molecule of the conjugate of the present invention may be any protein, peptide or fragment thereof having the ability to act as a ligand for a receptor present on a surface of a particular cell so that the targeting molecule can function to target the conjugate to the desired cell. The desired cell may be an infected cell, a bacterial or other type of pathogenic cell, a transformed cell, a tumor cell, a metastatic cell, and the like. The targeting molecule thus is a ligand for a receptor present on an infected cell, bacterial cell, tumor cell, etc., wherein the receptor is uniquely expressed or overexpressed on the surface of the infected cell, bacterial cell, tumor cell, etc., and thus “marks” the cell as being an infected cell, bacterial cell, tumor cell, etc.

The targeting molecule may be a true ligand for the cell surface receptor and bind in a binding groove of the receptor. Alternatively, the targeting molecule may be an antibody or fragment thereof raised against an epitope comprising a portion of the cell surface receptor, and capable of binding to the receptor when it is expressed on the surface of a cell of interest.

In one embodiment, the targeting molecule may be a heavy chain portion of an immunoglobulin product. The immunoglobulin product can be defined as: A polypeptide, protein or multimeric protein containing at least the immunologically active portion of an immunoglobulin heavy chain and is thus capable of specifically combining with an antigen. Exemplary immunoglobulin products are an immunoglobulin heavy chain, immunoglobulin molecules, substantially intact immunoglobulin molecules, any portion of an immunoglobulin that contains the paratope, including those portions known in the art as Fab fragments, Fab′ fragment, F(ab′)₂ fragment and Fv fragment. The antibody or fragment thereof may be produced by any means known in the art, and may be modified by any means known in the art. Alternatively, the targeting molecule may be any protein or peptide capable of binding to the epitope recognized by the antibody.

Many antibodies and other targeting approaches have been raised against tumor associated antigens (TAAs) that have recently been identified. Examples of such antibodies are disclosed in, for example but not by way of limitation, U.S. Pat. No. 5,250,297, issued to Grauer et al. on Oct. 5, 1993; U.S. Pat. No. 5,411,884, issued May 2, 1995 to Hellstrom et al.; U.S. Pat. No. 5,597,707, issued Jan. 28, 1997 to Marken et al.; U.S. Pat. No. 5,639,621, issued Jun. 17, 1997 to Bosslet et al.; U.S. Pat. No. 5,665,357, issued to Rose et al. on Sep. 9, 1997; U.S. Pat. No. 6,090,789, issued Jul. 18, 2000 to Danishefsky et al.; U.S. Pat. No. 6,596,503, issued Jun. 22, 2003 to Wennerberg et al.; and U.S. Pat. No. 6,926,896, issued Aug. 9, 2005 to Bosslet et al.; the contents of each of which is hereby expressly incorporated herein by reference. Since the majority of these TAAs are self antigens resulting from the overexpression of normal cellular genes, the ability to induce an immune response has been severely inhibited by self-tolerance. The ability of the present invention to conjugate such targeting molecules to an immunostimulatory molecule such as TLR ligands like CpG-ODN provides a unique and novel method of therapy for cancer.

In addition to antibodies against tumor associated antigens, antibodies raised against infectious diseases and/or autoimmune disorders may also be used in accordance with the present invention. In yet another embodiment, the targeting molecule may be a TCR mimic as described in published application US 2006/034850, published to Weidanz et al. on Feb. 16, 2006, the contents of which is hereby expressly incorporated herein by reference.

While the use of antibodies or fragments thereof has been disclosed herein previously, it is to be understood that any ligand for a cell surface receptor present on a cell of interest, wherein the ligand can be conjugated to a TCR ligand as described herein, may be utilized the targeting molecule in accordance with the present invention. Examples of targeting molecules that function as described herein are extremely numerous and will be easily envisioned by a person having ordinary skill in the art. Therefore, no further explanation of targeting molecules that can be utilized in accordance with the present invention is believed necessary.

The components of the conjugate of the present invention may be attached together via a cleavable linkage. The cleavable linkage between the targeting molecule and the TLR ligand may be any cleavable linkage known in the art that can function in accordance with the present invention. The cleavable linkage must be stable enough to allow a substantial amount of the conjugate to be administered at a site remote from the desired target cell and remain intact until delivered to the targeted site, such as a tumor. The cleavable nature of the linkage allows the bond between the TLR ligand and the targeting molecule to be broken after a certain period of exposure to physiological conditions. Any cleavable linkages known in the art that can function as described herein above may be utilized in the conjugates of the present invention. For example but not by way of limitation, a disulfide bond may be utilized as the cleavable linker; alternatively, peptide linkers may be used, and such molecules may then be digested by, for example, extracellular proteases, to cleave the bond and release the TLR ligand.

Alternatively, the components of the conjugate of the present invention may be attached together via a non-cleavable linkage. Any non-cleavable linkages known in the art that can function as described herein above may be utilized in the conjugates of the present invention. An example of a non-cleavable linkage that may be utilized in accordance with the present invention is a hydrazone linkage.

Methods of attaching proteins and oligonucleotides to form conjugates have been known previously (see, for example U.S. Pat. No. 6,942,972, issued to Farooqui et al. on Sep. 13, 2005; the contents of which are hereby expressly incorporated herein by reference). However, all of the methods of the prior art required a non-cleavable linkage between the two components of the conjugate. This is significant, as the two components of the conjugate each bind to receptors on different cells, and therefore the ability to cleave the conjugate frees the two molecules to act on these different cells when the two cells are present in close proximity. As demonstrated herein below, a conjugate constructed with a non-cleavable bond does not possess the same activity as a conjugates constructed with a cleavable bond of the present invention.

In addition, the present invention is the first to describe the conjugation of immunostimulatory CpG oligonucleotides to proteins for purposes of targeting.

The present invention further includes a method of inducing a targeted inflammatory response. In the method, the conjugate described above is provided, and an effective amount of the conjugate is administered to induce an inflammatory response at a targeted location, such as a tumor. In one embodiment, the conjugate is administered at a site remote from the targeted location.

The present invention also includes a method of treating a disease, including but not limited to, a cancer, tumor, infectious disease, or automimmune disease, by targeted CpG ODN delivery via conjugation to an antibody, thereby inducing an immune response. In addition to its antigen binding activity, it is known that the CpG-antibody conjugate affects the tumor microenvironment by inducing an immune response. Specifically, the conjugated proteins: (1) activate antigen presenting cells; (2) induce the secretion of proinflammatory cytokines and chemokines; (3) reduce the number of T regulatory cells; (4) alter the number of myeloid suppressor cells (the higher number of myeloid suppressor cells may be the result of conversion of M1 type macrophages to M2 type macrophages); (5) activate and attract NK cells at the tumor site; and (6) activate and attract CD4+ and CD8+ T cells at the tumor site.

The present invention also includes a vaccine comprising the conjugates described hereinabove, such as but not limited to, a CpG-antibody conjugate. The CpG-antibody conjugate is demonstrated herein as activating T cells and thus would be effective as a vaccine. The present invention also includes methods of making such vaccine, as described herein or otherwise known in the art. In addition, the present invention further includes methods of using such vaccine to elicit an immune response by administering an effective amount of the vaccine to a subject.

An Example is provided herein below. However, the present invention is to be understood to not be limited in its application to the specific experimentation, results and laboratory procedures described herein; rather, the Example is simply provided as one of various embodiments and is meant to be exemplary, not exhaustive.

### EXAMPLE

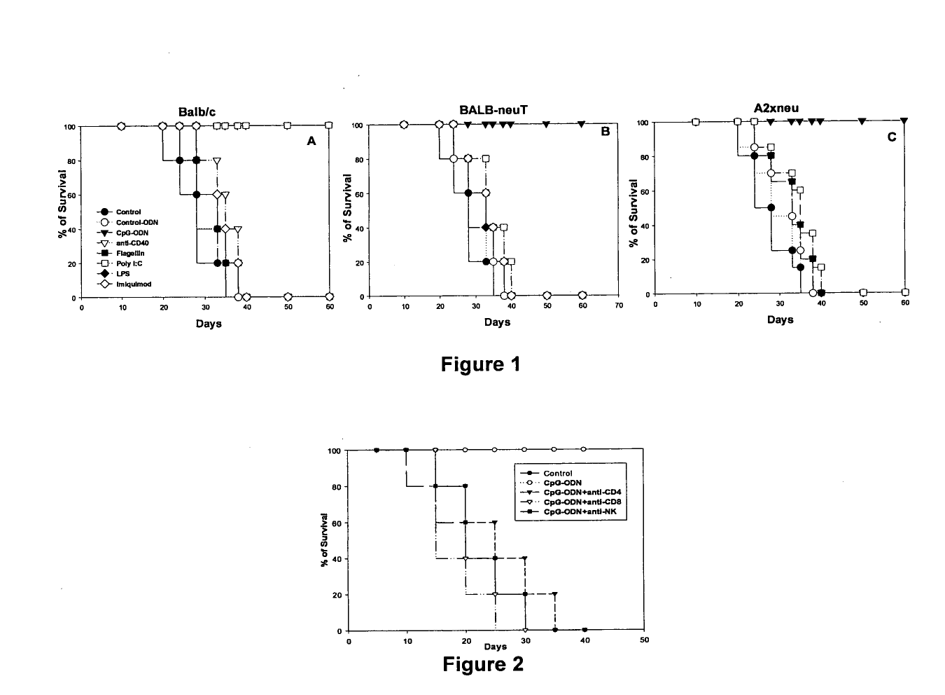

To determine which strategy or adjuvant would be the most effective in order to activate APC in Her-2/neu mice, antitumor immune responses were compared by targeting APCs after injecting a TNFR ligand like anti-CD40 agonist mAb and TLR ligands such as Poly I:C (TLR-3), LPS (TLR-4), flagellin (TLR-5), imiquimod (a soluble form of this compound was obtained directly from 3M pharmaceuticals) (TLR-7) and CpG-ODN (TLR-9). For these experiments, Balb/c (non-tolerant) and BALB-neuT (tolerant) mice implanted with TUBO cells and A2×neu mice implanted with N202.A2 cells were used (it is important to remember that TUBO cells are tumorigenic in Balb/c and BALB-neuT mice, while N202.A2 cells are tumorigenic only in A2×neu mice but not in A2×FVB mice). It is well established that the injection of these ligands results in the activation of DCs, monocytes/macrophages, and B cells, and as a consequence of the activation of these cells, NK cell and T cell responses are stimulated. Multiple reports have also demonstrated that injections of these ligands are capable of activating specific antitumor immune responses, resulting in the rejection of tumors (Janeway, 1989; Means et al., 2000; Kaisho et al., 2002; Takeda et al., 2003; Aderem et al., 2000; Means et al., 2000; Takeuchi et al., 2002; Gerwitz et al., 2001; Krieg, 2002; Yamamoto et al., 1992; Sun et al., 1998; and Hartmann et al., 2000). Balb/c and BALB-neuT mice were implanted s.c. with 10⁶ TUBO cells, and A2×neu mice were implanted s.c. with 10⁶ N202.A2 cells on day zero. On day 10, animals started treatment with s.c. injections of anti-CD40 mAb, Poly I:C, LPS, flagellin, imiquimod, CpG-ODN and control-ODN (as a control) in the opposite flank from where the tumor was injected, three times a week (20 μg/injection) for three weeks. Surprisingly, no antitumor effect was observed under these conditions (data not shown). It was decided to test whether intratumoral (i.t.) injection of TLR ligands would result in the induction of an antitumor response. The effect of i.t. injections of anti-CD40 mAb, Poly I:C, LPS, flagellin, imiquimod, CpG-ODN and control-ODN (as a control) three times a week (20 μg/injection) for three weeks was tested. As shown in FIG. 1, i.t. injections of CpG-ODN completely rejected the tumor in Balb/c, (FIG. 1A), BALB-neuT (FIG. 1B) and A2×neu mice (FIG. 1C). An important observation is that i.t. injections of Poly I:C also induced the rejection of tumors in Balb/c mice (FIG. 1A) but not in BALB-neuT or A2×neu mice (FIG. 1B-C). Treatment with anti-CD40 mAb, LPS, imiquimod or flagellin did not have any effect in controlling the tumor growth in these animals.

CpG-ODN antitumor immune responses depend on CD4+ T cell, CD8+ T cell and NK cell responses. It is possible that the initial immune response induced by CpG-ODN is mediated through the activation of antigen presenting cells (APC), which is unspecific in the beginning, and can subsequently activate other cells, such as NK cells, or prime a tumor-specific immune response. Unfortunately there are no available specific antibodies to eliminate subsets of APCs and directly evaluate the antitumor effect of these cells. Therefore, it was evaluated whether the antitumor response depends on CD4+ T cells, CD8+ T cells or NK cells. BALB-neuT mice were treated with i.p. injections of anti-CD4, anti-CD8 or anti-asialoGM1 antibodies (anti-NK Ab) (300 μg/injection) twice a week starting one week prior to tumor implantation and throughout the duration of the experiment. BALB-neuT mice were implanted with 10⁶ TUBO cells, and on day 10, animals started treatment with CpG-ODN as described above. As shown in FIG. 2, depletion of CD4+ T cells, CD8+ T cells and NK cells abrogates the anti-tumor response, indicating that these cells are critical for the rejection of the tumor after CpG-ODN injection. Similar results were observed with Balb/c and A2×FVB-neu mice (data not shown).

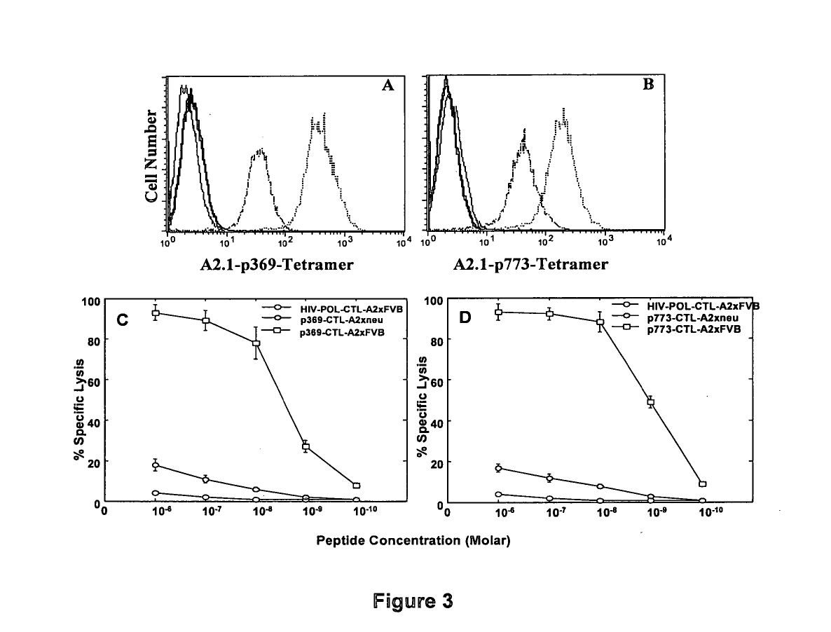

To test the effect that tolerance has on the immune response against Her-2/neu antigens, Her-2/neu transgenic mice were used in which the Her-2/neu protooncogene was expressed in the mammary tissue under the control of the MMTV promoter (FVB-neu transgenic mice) (Muller et al., 1988; and Guy et al., 1992). In order to analyze peptide specific responses against the A2.1/Her-2/neu p369-377 and p773-782 restricted epitopes that have previously been identified by the inventor (Lustgarten et al., 1997), the neu mice were crossed with A2.1/Kb transgenic mice (A2×neu) (Lustgarten et al., 2004). A2.1/Kb mice crossed with non-transgenic FVB mice (A2×FVB) were used as a control. Both the A2×neu and A2×FVB mice were immunized with the p369 and p773 peptides. As shown in FIG. 3, tetramer staining and cytotoxic activity, CTLs obtained from A2×neu mice demonstrated significantly lower affinity for both the p369 and p773 peptides when compared to CTLs from A2×FVB mice (Lustgarten et al., 2004; and Cuadros et al., 2005). The CTLs from A2×neu mice required more peptide, at least 100 fold, to achieve comparable lysis than CTLs from A2×FVB mice. A restricted HLA-A2.1/HIV-POL-CTL was used as a control, demonstrating that recognition of the neu-restricted-CTLs was specific. The results strongly indicate that T cells from A2×neu mice are hypo-responsive to neu antigens, and that these animals lack high avidity T cells for A2/neu immunodominant epitopes.

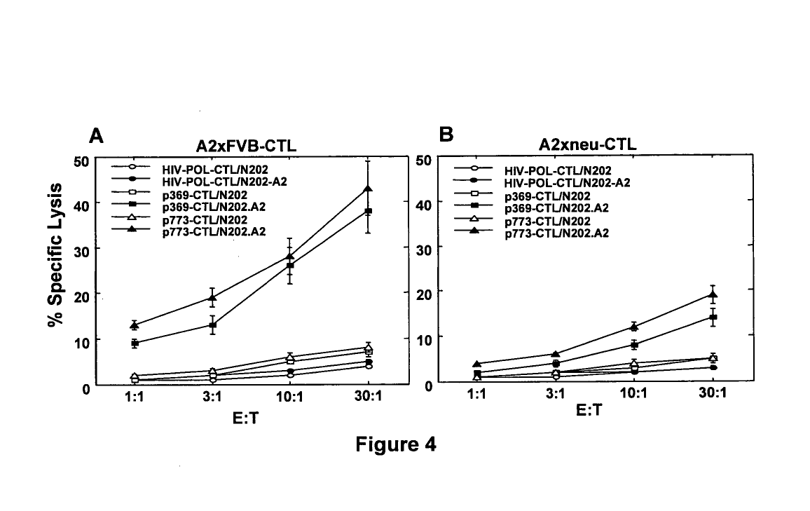

To evaluate in vivo antitumor responses in A2×neu mice, syngeneic tumor cell lines (N202.A2 and N202) were established from a spontaneous tumor in A2×neu or neu mice, respectively (Lustgarten et al., 2004; and Cuadros et al., 2005). The N202.A2 cells expressed A2.1 and Her-2/neu molecules (Lustgarten et al., 2004). It was tested whether the CTLs from A2×neu mice were able to recognize the N202.A2 cells. The CTLs from A2×neu and A2×FVB mice were incubated with ⁵¹Cr labeled N202 (a cell established from an FVB-neu mouse that does not express A2.1 molecules) and N202-A2. As shown in FIG. 4, CTLs from A2×neu mice were capable of recognizing the N202-A2 targets, albeit at significantly lower levels than CTLs from A2×FVB mice. The CTLs did not recognize N202 cells, indicating that these CTL recognized A2-neu restricted antigens expressed on tumor cells. The ability of N202.A2 cells to grow in A2×neu and A2×FVB mice was also measured. The N202.A2 cells formed tumors in A2×neu mice, but were unable to grow in A2×FVB mice (Lustgarten et al., 2004; and Cuadros et al., 2005). These data are in agreement with the in vitro results that neu mice are tolerant to neu antigens.

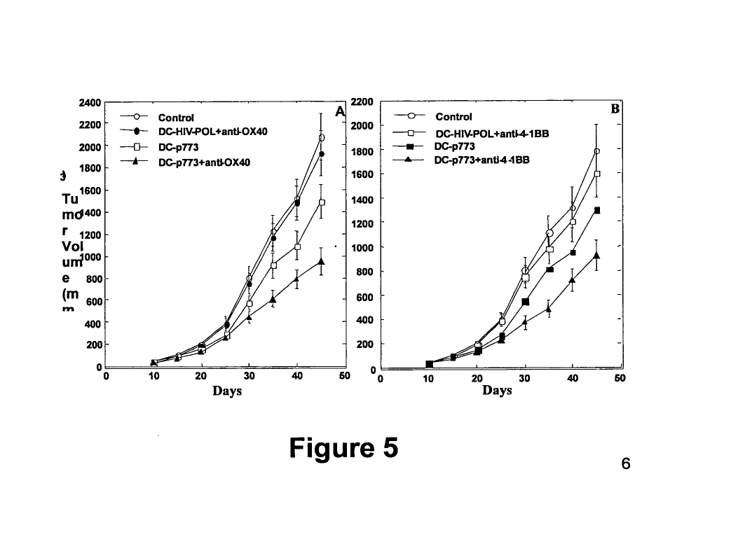

The preceding results demonstrated that CTLs derived from A2×neu-mice have the capacity to recognize and kill the N202.A2 tumor cells in vitro and in vivo, albeit with low efficiency. Next, it was desired to evaluate whether immunization of A2×neu mice would induce an immune response capable of delaying or rejecting the growth of an established tumor. In the last few years OX-40 and 4-1 BB have gained importance as co-stimulatory molecules capable of expanding the immune responses and enhancing the antitumor immune responses of animals with established tumors (Laderach et al., 2002; Weinberg, 2002; Taraban et al., 2002; and Kim et al., 2001). It was also evaluated whether the combination of DCs-based vaccine and anti-OX40 or anti-4-1 BB would stimulate a stronger antitumor response in A2×neu mice. As shown in FIG. 5, DC-based vaccines plus anti-OX40 or anti-4-1 BB induced a stronger protective antitumor response, resulting in an ˜35-45% tumor growth inhibition, while DC-based vaccination in the absence of anti-OX40 or anti-4-1 BB mAb only inhibited-20-25% of the tumor growth (Cuadros et al., 2003; Lustgarten et al., 2004; and Cuadros et al., 2005). Similar results were found with the p369 peptide (data not shown).

The results described previously demonstrate that when the immune responses of Her-2/neu mice immunized with DCs pulsed with a soluble neu protein or with apoptotic tumor cells were compared, the antitumor response showed that Her-2/neu mice vaccinated with DCs pulsed with Her-2/neu antigens retarded the tumor growth; however, vaccination with DCs pulsed with apoptotic tumor cells induced a stronger antitumor effect. Additionally, in order to generate a stronger antitumor response, animals needed to be immunized multiple-times in combination with the co-stimulatory agonist anti-OX40 and anti-4-1 BB mAb (Cuadros et al., 2005). Although the ability to significantly enhance the antitumor immune response in neu mice with the use of DC-vaccination in combination with costimulatory molecules was demonstrated, vaccination therapy was not sufficient for the complete elimination of tumors (Lustgarten et al., 2004; and Cuadros et al., 2005).

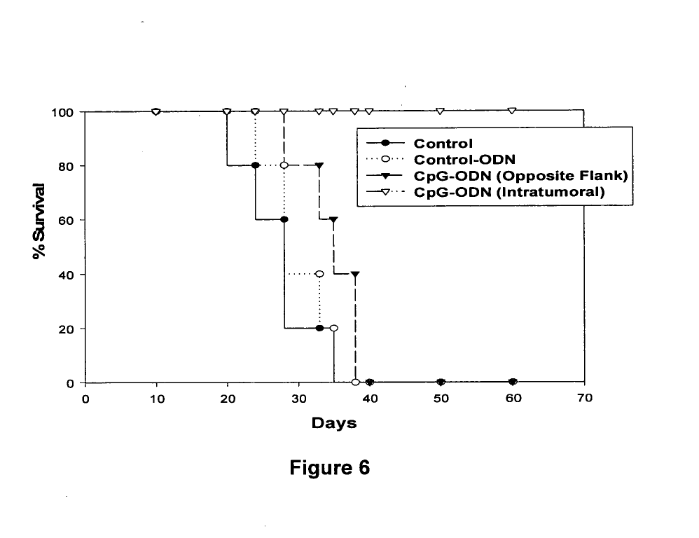

It is well established that CpG-ODN are potent adjuvants for enhancing immune responses (Heit et al., 2005; Davila et al., 2003; Davila et al., 2000; and Heckelsmiller et al., 2002). The majority of studies evaluating CpG-ODN have used CPG-ODN as an adjuvant to boost T cell responses after antigen vaccination (Hiraoka et al., 2004; and Miconnet et al., 2002). One of the consequences of injecting CpG-ODN is the activation of DCs, monocytes/macrophages, B cells and NK cells. Based on the accumulative evidence that CpG-ODN strongly activates an immune response, it was decided to test the effect of injecting CpG-ODN intratumorally. Neu mice were implanted s.c. with 10⁶ N202 cells on day zero. On day seven (as in FIG. 5), animals were treated with i.t. injections of CpG-ODN three times a week (20 μg/injection) for three weeks. Control-ODN (i.t. injection) and injections of CpG-ODN in the opposite flank from where the tumor was injected were used as a control. As shown in FIG. 6, i.t. injections of CpG-ODN completely rejected the tumor, while no antitumor effect was observed in neu mice injected intratumorally with control-ODN or systemic injection of CpG-ODN. These results demonstrate the potent effect of injecting CpG-ODN at the tumor site. For the first time, the results show that i.t. injections of CpG-ODN circumvent tolerance and are a good strategy for inducing tumor rejection in neu mice.

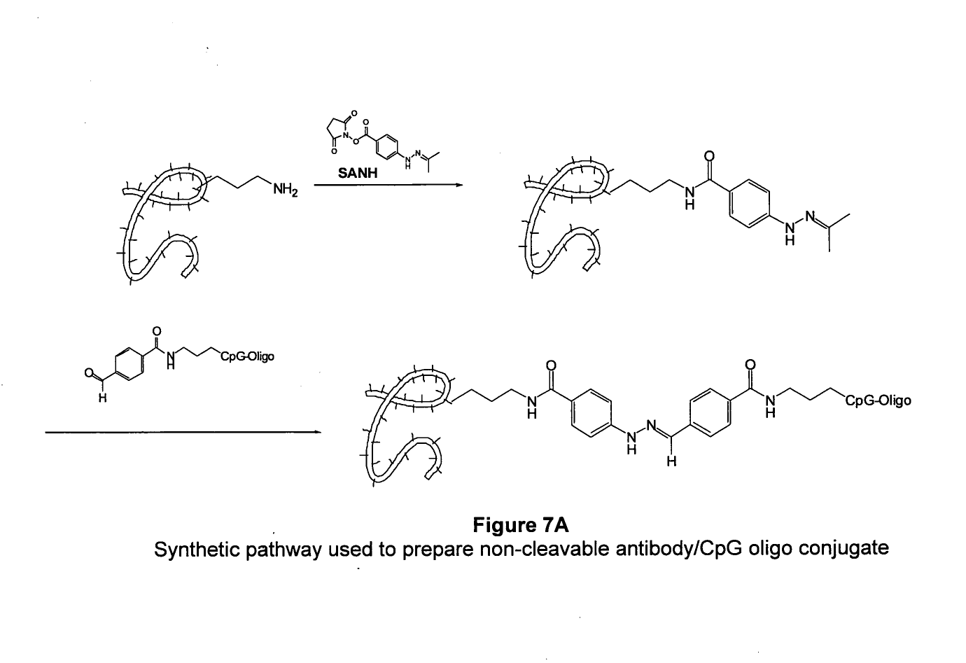

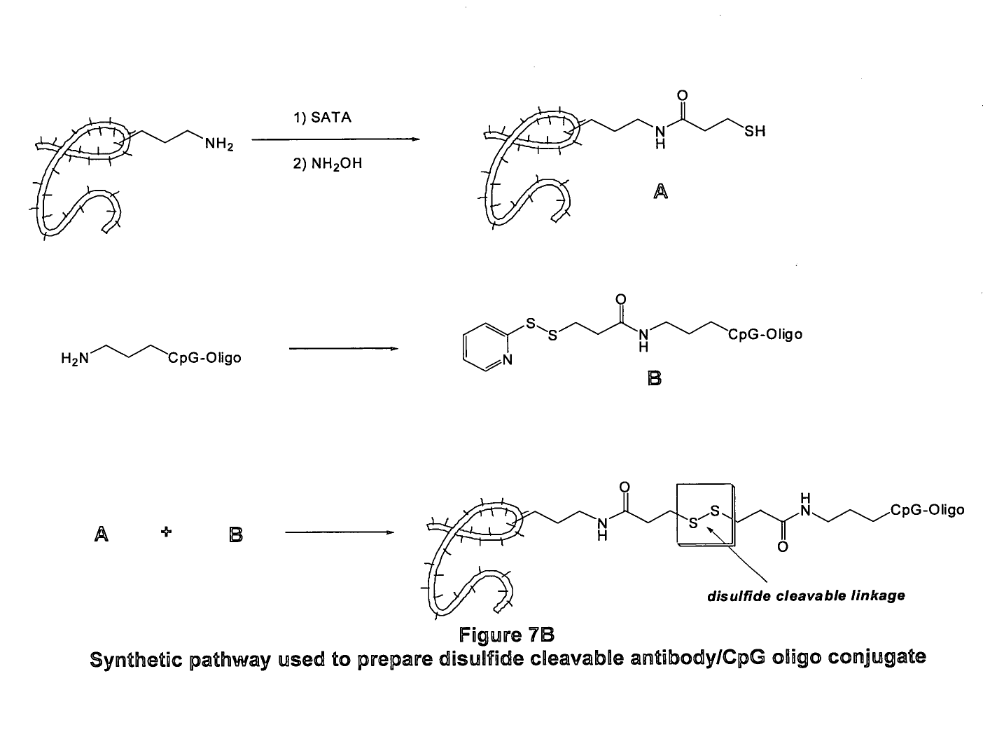

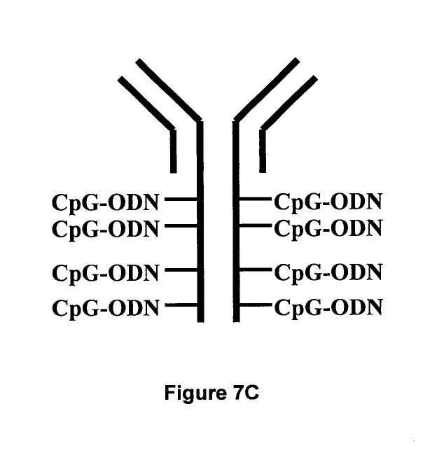

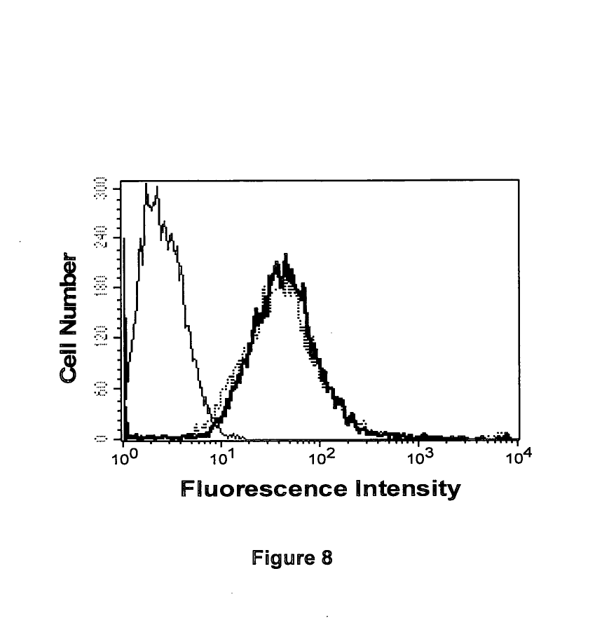

The results presented herein clearly demonstrated that CpG-ODN is the most effective adjuvant for inducing an antitumor response in tolerant hosts. Critical to induction of antitumor responses in A2×neu or BALB-neuT mice is that the CpG-ODN should be injected at the site of the tumor. The major drawback of this approach, however, is that not all tumors will be physically available for intratumoral injections. Therefore, the generation of a targeted-CpG-ODN for universal use will be more practical. In order to target the CpG-ODN to the tumor, it was decided to produce a fusion protein between an antibody directed against the rat neu molecule and CpG-ODN. The CpG-ODN was conjugated to the anti-neu mAb (7.16.4) via either a cleavable or non-cleavable bond. Anti-neu-CpG-ODN generated with a non-cleavable bond was produced by the reaction pathway shown in FIG. 7A and contained a hydrazone linkage between the Anti-neu antibody and the CpG-ODN. Anti-neu-CpG-ODN generated with a cleavable bond was produced by the reaction pathway shown in FIG. 7B and contained a disulfide cleavable linkage. For the results described herein below with relation to FIGS. 8-13, the Anti-neu-CpG-ODN generated with a cleavable bond was utilized. A schematic representation of the Anti-neu-CpG-ODN molecule is shown in FIG. 7C.

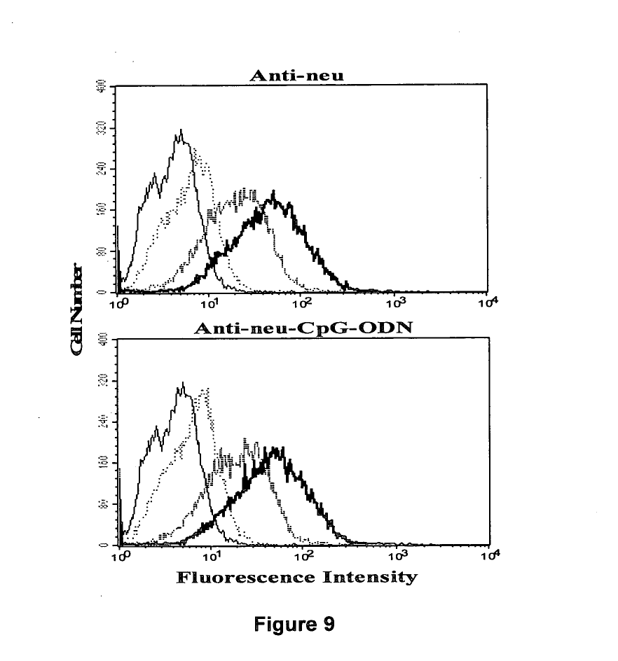

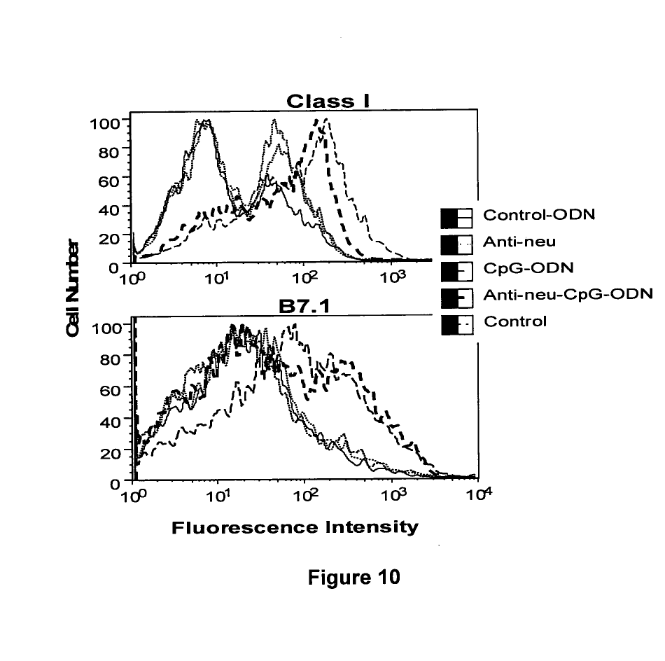

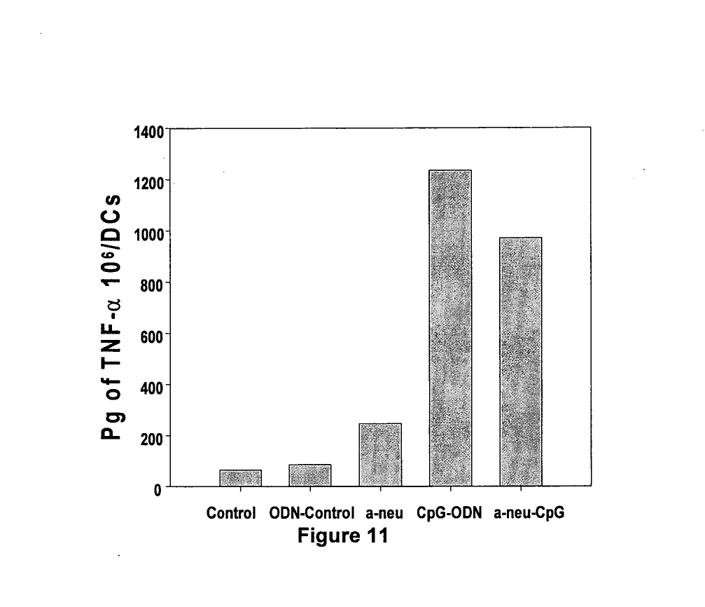

Characterization of anti-neu-CpG-ODN conjugated molecules. It was tested whether the anti-neu-CpG-ODN retained its ability to bind Her-2/neu+ cells and its capacity to stimulate and induce the maturation and activation of DCs. Anti-neu and anti-neu-CpG-ODN antibodies were diluted 1/10, 1/100 and 1/1000, and as shown in FIGS. 9 and 10, the anti-neu-CpG-ODN binds equally to TUBO (FIG. 9) or N202.A2 (FIG. 10) cells as does the anti-neu mAb, thus indicating that the anti-neu-CpG-ODN retains its affinity/avidity for the neu antigen present on tumor cells. DCs derived from bone marrow were incubated in the presence of 0.5 μg (for TUBO; FIG. 9) or 1 μg (for N202.A2; FIG. 10) of CpG-ODN, anti-neu-CpG-ODN, control-ODN or anti-neu mAb overnight. The next day, DCs were recovered and analyzed for the up-regulation of cellular markers. As shown in FIGS. 9 and 10, the anti-neu-CpG-ODN induced the activation of DCs by increasing the levels of expression of class I and B7.1 molecules (higher levels of class II and B7.2 were also observed after CpG-ODN and anti-neu-CpG-ODn stimulation, data not shown) with the same efficiency as CpG-ODN, while stimulation with control-ODN or anti-neu mAb showed no stimulatory effect. The secretion of TNF-α was also examined following stimulation of DCs with anti-neu-CpG-ODN, CpG-ODN, control-ODN or anti-neu mAb. DCs treated with anti-neu-CpG-ODN produced similar amounts of TNF-α as did DCs treated with CpG-ODN (FIG. 11). However, no production of TNF-α was detected after treatment with control-ODN or anti-neu mAb. These results demonstrated that the anti-neu-CpG-ODN retained its dual capacity of: (1) binding to Her-2/neu+tumor cells; and (2) activating DCs.

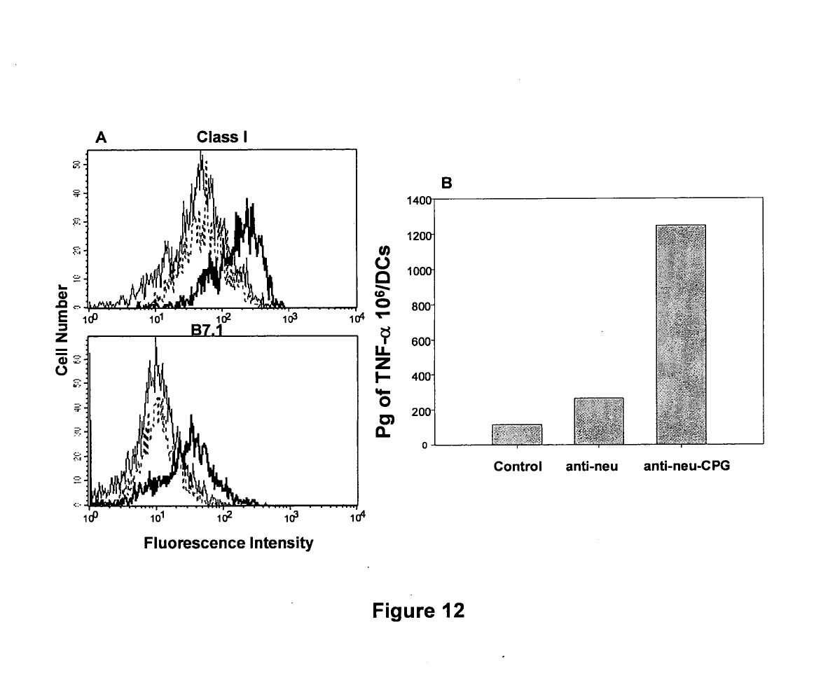

Stimulatory effect of anti-neu-CpG-ODN bound onto tumor cells. The ability of anti-neu-CpG-ODN bound onto tumors to stimulate DCs was also tested. Cultured N202 cells (5×10⁵/well) bound to 24 well plates (these cells are attached to the plastic) were incubated with anti-neu mAb, anti-neu-CpG-ODN (1 μg), or no antibody for one hour and washed twice to remove free antibodies. Cultured DCs (5×10⁵/well) were then added and incubated overnight. The next day, DCs were recovered (DCs do not become attached to the plastic) and stained to evaluate the expression of cellular markers. As shown in FIG. 12, only N202 cells incubated with anti-neu-CpG-ODN stimulated the DCs, increasing the expression of class I and B7.1 (FIG. 12A) molecules (the levels of B7.2 and class II molecules were also increased, data not shown) and inducing the secretion of TNF-α (FIG. 12B). Taken together, these results demonstrate that anti-neu-CpG-ODN bound onto tumors can stimulate and activate DCs, indicating that the use of anti-neu-CpG-ODN could be a feasible strategy for treating targeted tumors.

As a note, treatment of neu-tumor bearing mice with the anti-neu 7.16.4. mAb (twice a week, 100 μg/injection) induces a 20-25% tumor growth inhibition. Similar data was found where treatment with herceptin (anti-human-neu mAb) delays tumor growth in breast cancer patients. As such, it is necessary to identify active immunization conditions capable of inducing a long-term immunity to Her-2/neu antigens resulting in the rejection of tumors. Therefore, the use of the anti-neu-CpG-ODN strategy could have the inhibitory benefit of the anti-neu mAb and be able to activate antitumor immune responses.

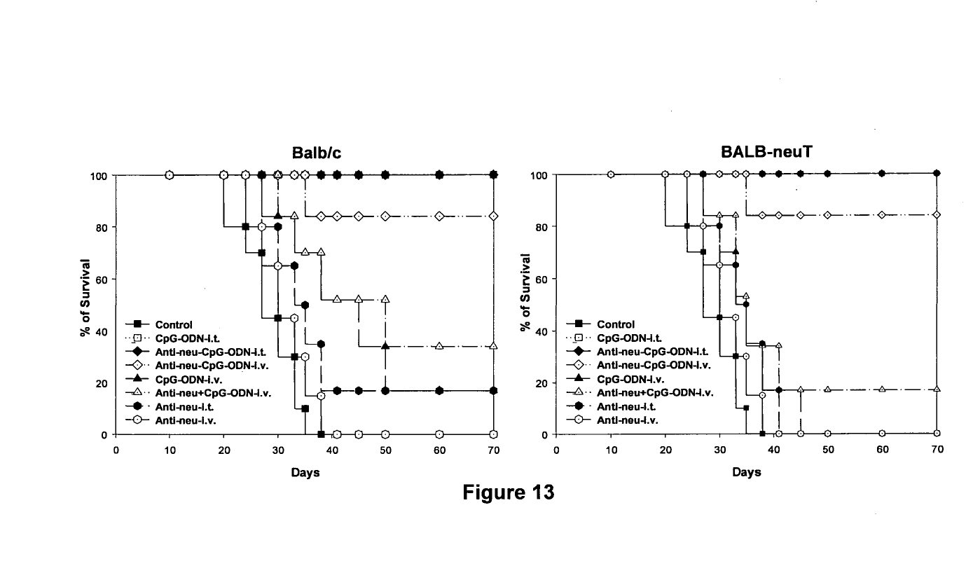

The results presented herein above indicate that i.t. injections of CpG-ODN induce the elimination of tumors on neu mice. Therefore, it was desired to evaluate whether the anti-neu-CpG-ODN would have the same antitumor effect. TUBO cells (1×10⁶) were implanted s.c. on day zero on Balb/c and BALB-neuT mice. On day 10, animals were injected i.v. with 50 μg/injection of anti-neu-CpG-ODN twice a week for three weeks. Anti-neu-CpG-ODN was injected i.t. to test and make sure that anti-neu-CpG-ODN could induce the rejection of tumors. The following groups were also included as controls: (1) CpG-ODN injected i.t.; (2) CpG-ODN injected i.v.; (3) CpG-ODN+anti-neu injected i.v.; (4) anti-neu injected i.t.; and (5) anti-neu injected i.v. As shown in FIG. 13, i.t. injections of CpG-ODN and anti-neu-CpG-ODN completely rejected the tumors in both Balb/c and BALB-neuT mice, and 5/6 BALB-neuT (84%) and 5/6 Balb/c (84%) mice injected i.v. with anti-neu-CpG-ODN rejected the tumor. Importantly, animals that did not reject tumors after i.v. injections with anti-neu-CpG-ODN exhibited significantly delayed tumor growth when compared to control animals. It was observed that 2/6 Balb/c mice (34%) injected i.v. with CpG-ODN+anti-neu rejected the tumors and 1/6 (17%) of Balb/c injected i.v. with CpG-ODN rejected the tumor. BALB-neuT mice injected i.v. with CpG-ODN (+/−anti-neu) did not develop an antitumor response or control the tumor growth in these animals. A delay in tumor growth was not observed in animals injected with anti-neu mAb, and only 1/6 (16%) Balb/c mice injected i.t. with anti-neu mAb survived. These data confirmed that anti-neu-CpG-ODN is a functional molecule in vivo, delivering the CpG-ODN to the tumor site and resulting in the rejection of tumors. The antitumor response induced by anti-neu-CpG-ODN is also CD4+ and CD8+ T cell and NK cell dependent (data not shown) as was shown with soluble CpG-ODN (FIG. 2). To truly observe the potency and efficacy of the anti-neu-CpG-ODN conjugated molecule, it is important to know that 1 μg of antibody contains 0.04 μg of CpG-ODN. Therefore, when 50 μg/injection of anti-neu-CpG-ODN was injected, only 0.2 μg/injection of total CpG-ODN was injected. An evaluation of injecting 0.2 μg/injection of CpG-ODN i.t. three times a week for three weeks was performed, and Balb/c or BALB-neuT mice succumbed to the tumor (data not shown). Taken together, these results have important clinical implications: (1) These results are in agreement with data generated from in vitro experiments indicating that low concentrations of CpG-ODN (0.1 μg or lower) are sufficient to activate APCs; (2) the CpG-ODN linked to the antibody most probably remains for longer periods of time at the tumor site when compared to soluble CpG-ODN; (3) due to the length of time that the antibody-CpG-ODN remains at the tumor site, low concentrations of CpG-ODN are sufficient to activate an immune response; however, high concentrations of CpG-ODN are needed when it is injected at the tumor site as a soluble molecule; this may suggest that fewer or less frequent injections of antibody-CpG-ODN might be needed; (4) it has been demonstrated that high doses of CpG-ODN could have toxic side effects. Therefore, the use of anti-neu-CpG-ODN will have clinical benefits such as reducing the possible side effects of injecting high doses of CpG-ODN. These results are very encouraging and demonstrate the proof of concept that antibody-CpG-ODN conjugated molecules are functional in vitro and in vivo and that they can serve as a new strategy for fighting cancer. Furthermore, based on the data presented, anti-neu-CpG-ODN is superior to soluble CpG-ODN based on the dose applied. One of the methods of the presently claimed and disclosed invention is therefore to use anti-neu-CpG-ODN molecules to control primary and disseminated tumors, wherein the effective amount of the anti-neu-CpG-ODN molecules required for an antitumor effect is lower than the effective amount required of either CpG-ODN or anti-neu alone to have an antitumor effect.

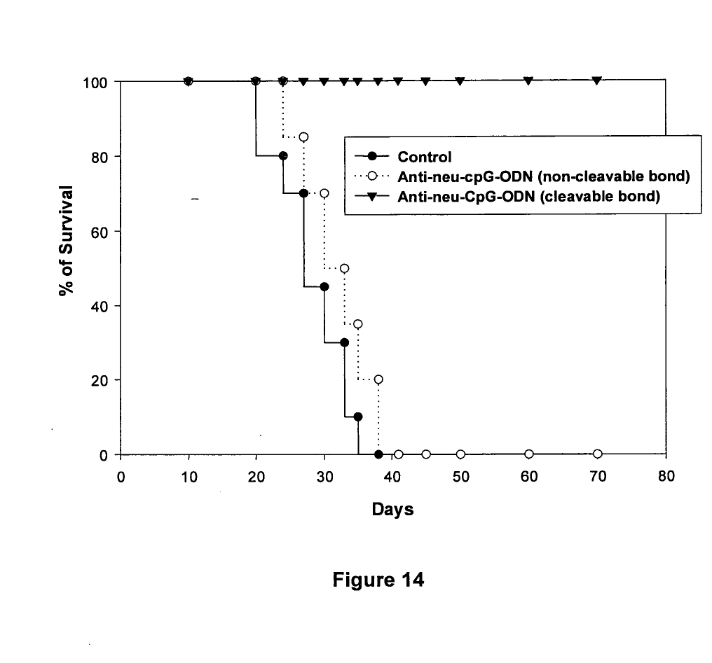

Evaluation of anti-neu-CpG-ODN (cleavable bond) vs. anti-neu-CpG-ODN (non-cleavable bond). In determining the effectiveness of the conjugates of the present invention, the antitumor effects of anti-neu-CpG-ODN generated with a cleavable bond or non-cleavable bond were evaluated. Anti-neu-CpG-ODN generated with a non-cleavable bond was produced by the reaction pathway shown in FIG. 7A and contained a hydrazone linkage between the Anti-neu antibody and the CpG-ODN. Anti-neu-CpG-ODN generated with a cleavable bond was produced by the reaction pathway shown in FIG. 7B and contained a disulfide cleavable linkage. TUBO cells (1×10⁶) were implanted s.c. on day zero on Balb/c. Starting on day 10, animals were injected intratumorally with 30 μg/injection of each of the anti-neu-CpG-ODN molecules three times a week for three weeks. As shown in FIG. 14, only animals treated with the anti-neu-CpG-ODN containing the cleavable bond induced the rejection of tumors. No antitumor effect was observed with anti-neu-CpG-ODN containing the non-cleavable bond. This demonstrates that the cleavable nature of the linkage between the CpG oligonucleotide and the targeting molecule to which it is attached appears to be a necessary feature of the present invention.

It is known that CpG-ODN binds to the Toll Like receptor 9 (TLR-9). TLR-9 is only expressed intracellularly on antigen presenting cells (APCs). It is believed that the reason that the anti-neu-CpG-ODN containing the cleavable bond works is because the CpG-ODN is cleaved or released from the antibody and then is acquired by APCs, resulting in their activation. In contrast, with the anti-neu-CpG-ODN containing the non-cleavable bond, the CpG-ODN is not released from the antibody and therefore does not stimulate an immune response.

Thus, in accordance with the present invention, there has been provided a method of producing conjugates effective in inducing an immune response, as well as methods of producing and using same, that fully satisfies the objectives and advantages set forth herein above. Although the invention has been described in conjunction with the specific drawings, experimentation, results and language set forth herein above, it is evident that many alternatives, modifications, and variations will be apparent to those skilled in the art. Accordingly, it is intended to embrace all such alternatives, modifications and variations that fall within the spirit and broad scope of the present invention.

### REFERENCES

The following references, to the extent that they provide exemplary procedural or other details supplementary to those set forth herein, are specifically incorporated herein by reference.
Acres, B, Paul, S, Haegel-Kronenberger, H, Calmels, B, and Squiban, P Therapeutic cancer vaccines. Curr Opin Mol Ther, 2004; 6(1): 40-47.
Aderem, A and Ulevitch, R J Toll-like receptors in the induction of the innate immune response. Nature, 2000; 406(6797): 782-787.
Ballas, Z K, Rasmussen, W L, and Krieg, A M Induction of NK activity in murine and human cells by CpG motifs in oligodeoxynucleotides and bacterial DNA. J Immunol, 1996; 157(5): 1840-1845.
Bargmann, C I, Hung, M C, and Weinberg, R A Multiple independent activations of the neu oncogene by a point mutation altering the transmembrane domain of p185. Cell, 1986; 45(5): 649-657.
Barton, G M and Medzhitov, R Control of adaptive immune responses by Toll-like receptors. Curr Opin Immunol, 2002; 14(3): 380-383.
Beloeil, L, Tomkowiak, M, Angelov, G, Walzer, T, Dubois, P, and Marvel, J In vivo impact of CpG1826 oligodeoxynucleotide on CD8 T cell primary responses and survival. J Immunol, 2003; 171(6): 2995-3002.
Bloom, M B, Perry-Lalley, D, Robbins, P F, et al. Identification of tyrosinase-related protein 2 as a tumor rejection antigen for the B16 melanoma. J Exp Med, 1997; 185(3): 453-459.
Brossart, P, Stuhler, G, Flad, T, et al. Her-2/neu-derived peptides are tumor-associated antigens expressed by human renal cell and colon carcinoma lines and are recognized by in vitro induced specific cytotoxic T lymphocytes. Cancer Res, 1998; 58(4): 732-736.
Carpentier, A F, Xie, J, Mokhtari, K, and Delattre, J Y Successful treatment of intracranial gliomas in rat by oligodeoxynucleotides containing CpG motifs. Clin Cancer Res, 2000; 6(6): 2469-2473.
Chen, Y T, Scanlan, M J, Sahin, U, et al. A testicular antigen aberrantly expressed in human cancers detected by autologous antibody screening. Proc Natl Acad Sci USA, 1997; 94(5): 1914-1918.
Cordaro, T A, de Visser, K E, Tirion, F H, Schumacher, T N, and Kruisbeek, A M Can the low-avidity self-specific T cell repertoire be exploited for tumor rejection? J Immunol, 2002; 168(2): 651-660.
Cuadros, C, Dominguez, A L, Frost, G I, Borgstrom, P, and Lustgarten, J Cooperative effect between immunotherapy and antiangiogenic therapy leads to effective tumor rejection in tolerant Her-2/neu mice. Cancer Res, 2003; 63(18): 5895-5901.
Cuadros, C, Dominguez, A L, Lollini, P L, et al. Vaccination with dendritic cells pulsed with apoptotic tumors in combination with anti-OX40 and anti-4-1 BB monoclonal antibodies induces T cell-mediated protective immunity in Her-2/neu transgenic mice. Int J Cancer, 2005.
Davila, E and Celis, E Repeated administration of cytosine-phosphorothiolated guanine-containing oligonucleotides together with peptide/protein immunization results in enhanced CTL responses with anti-tumor activity. J Immunol, 2000; 165(1): 539-547.
Davila, E, Kennedy, R, and Celis, E Generation of antitumor immunity by cytotoxic T lymphocyte epitope peptide vaccination, CpG-oligodeoxynucleotide adjuvant, and CTLA-4 blockade. Cancer Res, 2003; 63(12): 3281-3288.
Davila, E, Velez, M G, Heppelmann, C J, and Celis, E Creating space: an antigen-independent, CpG-induced peripheral expansion of naive and memory T lymphocytes in a full T-cell compartment. Blood, 2002; 100(7): 2537-2545.
Dranoff, G Cytokines in cancer pathogenesis and cancer therapy. Nat Rev Cancer, 2004; 4(1): 11-22.
El-Shami, K M, Tirosh, B, Popovic, D, et al. Induction of antitumor immunity by proteasome-inhibited syngeneic fibroblasts pulsed with a modified TAA peptide. Int J Cancer, 2000; 85(2): 236-242.
Feltkamp, M C, Smits, H L, Vierboom, M P, et al. Vaccination with cytotoxic T lymphocyte epitope-containing peptide protects against a tumor induced by human papillomavirus type 16-transformed cells. Eur J Immunol, 1993; 23(9): 2242-2249.
Geiger, T, Gooding, L R, and Flavell, R A T-cell responsiveness to an oncogenic peripheral protein and spontaneous autoimmunity in transgenic mice. Proc Natl Acad Sci USA, 1992; 89(7): 2985-2989.
Gewirtz, A T, Navas, T A, Lyons, S, Godowski, P J, and Madara, J L Cutting edge: bacterial flagellin activates basolaterally expressed TLR5 to induce epithelial proinflammatory gene expression. J Immunol, 2001; 167(4): 1882-1885.
Goldrath, A W and Bevan, M J Selecting and maintaining a diverse T-cell repertoire. Nature, 1999; 402(6759): 255-262.
Gusterson, B A, Gelber, R D, Goldhirsch, A, et al. Prognostic importance of c-erbB-2 expression in breast cancer. International (Ludwig) Breast Cancer Study Group. J Clin Oncol, 1992; 10(7): 1049-1056.
Guy, C T, Webster, M A, Schaller, M, Parsons, T J, Cardiff, R D, and Muller, W J Expression of the neu protooncogene in the mammary epithelium of transgenic mice induces metastatic disease. Proc Natl Acad Sci USA, 1992; 89(22): 10578-10582.
Hartmann, G and Krieg, A M Mechanism and function of a newly identified CpG DNA motif in human primary B cells. J Immunol, 2000; 164(2): 944-953.
Hartmann, G, Weeratna, R D, Ballas, Z K, et al. Delineation of a CpG phosphorothioate oligodeoxynucleotide for activating primate immune responses in vitro and in vivo. J Immunol, 2000; 164(3): 1617-1624.
Hausmann, S, Martin, M, Gauthier, L, and Wucherpfennig, K W Structural features of autoreactive TCR that determine the degree of degeneracy in peptide recognition. J Immunol, 1999; 162(1): 338-344.
Heckelsmiller, K, Beck, S, Rall, K, et al. Combined dendritic cell- and CpG oligonucleotide-based immune therapy cures large murine tumors that resist chemotherapy. Eur Immunol, 2002; 32(11): 3235-3245.
Heine, H and Lien, E Toll-like receptors and their function in innate and adaptive immunity. Int Arch Allergy Immunol, 2003; 130(3): 180-192.
Heit, A, Schmitz, F, O'Keeffe, M, et al. Protective CD8 T cell immunity triggered by CpG-protein conjugates competes with the efficacy of live vaccines. J Immunol, 2005; 174(7): 4373-4380.
Hiraoka, K, Yamamoto, S, Otsuru, S, et al. Enhanced tumor-specific long-term immunity of hemagglutinating [correction of hemaggluttinating] virus of Japan-mediated dendritic cell-tumor fused cell vaccination by coadministration with CpG oligodeoxynucleotides. J Immunol, 2004; 173(7): 4297-4307.
Houbiers, J G, Nijman, H W, van der Burg, S H, et al. In vitro induction of human cytotoxic T lymphocyte responses against peptides of mutant and wild-type p53. Eur J Immunol, 1993; 23(9): 2072-2077.
Huang, Q, Liu, D, Majewski, P, et al. The plasticity of dendritic cell responses to pathogens and their components. Science, 2001; 294(5543): 870-875.
Imler, J L and Hoffmann, J A Toll receptors in innate immunity. Trends Cell Biol, 2001; 11(7): 304-311.
Ioannides, C G, Fisk, B, Fan, D, Biddison, W E, Wharton, J T, and O'Brian, C A Cytotoxic T cells isolated from ovarian malignant ascites recognize a peptide derived from the HER-2/neu proto-oncogene. Cell Immunol, 1993; 151(1): 225-234.
Jakob, T, Walker, P S, Krieg, A M, Udey, M C, and Vogel, J C Activation of cutaneous dendritic cells by CpG-containing oligodeoxynucleotides: a role for dendritic cells in the augmentation of Th1 responses by immunostimulatory DNA. J Immunol, 1998; 161(6): 3042-3049.
Janeway, C A, Jr. Approaching the asymptote? Evolution and revolution in immunology. Cold Spring Harb Symp Quant Biol, 1989; 54 Pt 1(1-13.
Janku, F, Pribylova, O, Zimovjanova, M, et al. 4-years results of weekly trastuzumab and paclitaxel in the treatment of women with HER2/neu overexpressing advanced breast cancer: single institution prospective study. Bull Cancer, 2004; 91(10): E279-283.
Kaisho, T and Akira, S Toll-like receptors as adjuvant receptors. Biochim Biophys Acta, 2002; 1589(1): 1-13.
Kawai, K and Ohashi, P S Immunological function of a defined T-cell population tolerized to low-affinity self antigens. Nature, 1995; 374(6517): 68-69.
Kawakami, Y, Eliyahu, S, Delgado, C H, et al. Cloning of the gene coding for a shared human melanoma antigen recognized by autologous T cells infiltrating into tumor. Proc Natl Acad Sci USA, 1994; 91(9): 3515-3519.
Kim, J A, Averbook, B J, Chambers, K, et al. Divergent effects of 4-1BB antibodies on antitumor immunity and on tumor-reactive T-cell generation. Cancer Res, 2001; 61(5): 2031-2037.
Krieg, A M CpG motifs in bacterial DNA and their immune effects. Annu Rev Immunol, 2002; 20(709-760.
Kurt, R A, Whitaker, R, Baher, A, Seung, S, and Urba, W J Spontaneous mammary carcinomas fail to induce an immune response in syngeneic FVBN202 neu transgenic mice. Int Cancer, 2000; 87(5): 688-694.
Laderach, D, Movassagh, M, Johnson, A, Mittler, R S, and Galy, A 4-1 BB co-stimulation enhances human CD8(+) T cell priming by augmenting the proliferation and survival of effector CD8(+) T cells. Int Immunol, 2002; 14(10): 1155-1167.
Lee, K F, Simon, H, Chen, H, Bates, B, Hung, M C, and Hauser, C Requirement for neuregulin receptor erbB2 in neural and cardiac development. Nature, 1995; 378(6555): 394-398.
Liang, H, Nishioka, Y, Reich, C F, Pisetsky, D S, and Lipsky, P E Activation of human B cells by phosphorothioate oligodeoxynucleotides. J Clin Invest, 1996; 98(5): 1119-1129.
Lo, D, Freedman, J, Hesse, S, Palmiter, R D, Brinster, R L, and Sherman, L A Peripheral tolerance to an islet cell-specific hemagglutinin transgene affects both CD4+ and CD8+ T cells. Eur J Immunol, 1992; 22(4): 1013-1022.
Lurquin, C, Van Pel, A, Mariame, B, et al. Structure of the gene of tum- transplantation antigen P91A: the mutated exon encodes a peptide recognized with Ld by cytolytic T cells. Cell, 1989; 58(2): 293-303.
Lustgarten, J, Dominguez, A L, and Cuadros, C The CD8+ T cell repertoire against Her-2/neu antigens in neu transgenic mice is of low avidity with antitumor activity. Eur J Immunol, 2004; 34(3): 752-761.
Lustgarten, J, Theobald, M, Labadie, C, et al. Identification of Her-2/Neu CTL epitopes using double transgenic mice expressing HLA-A2.1 and human CD.8. Hum Immunol, 1997; 52(2): 109-118.
Machiels, J P, Reilly, R T, Emens, L A, et al. Cyclophosphamide, doxorubicin, and paclitaxel enhance the antitumor immune response of granulocyte/macrophage-colony stimulating factor-secreting whole-cell vaccines in HER-2/neu tolerized mice. Cancer Res, 2001; 61(9): 3689-3697.
Means, T K, Golenbock, D T, and Fenton, M J Structure and function of Toll-like receptor proteins. Life Sci, 2000; 68(3): 241-258.
Means, T K, Golenbock, D T, and Fenton, M J The biology of Toll-like receptors. Cytokine Growth Factor Rev, 2000; 11(3): 219-232.
Mercader, M, Bodner, B K, Moser, M T, et al. T cell infiltration of the prostate induced by androgen withdrawal in patients with prostate cancer. Proc Natl Acad Sci USA, 2001; 98(25): 14565-14570.
Miconnet, I, Koenig, S, Speiser, D, et al. CpG are efficient adjuvants for specific CTL induction against tumor antigen-derived peptide. J Immunol, 2002; 168(3): 1212-1218.
Muller, W J, Sinn, E, Pattengale, P K, Wallace, R, and Leder, P Single-step induction of mammary adenocarcinoma in transgenic mice bearing the activated c-neu oncogene. Cell, 1988; 54(1): 105-115.
Nieto, Y, Vredenburgh, J J, Shpall, E J, et al. Phase II feasibility and pharmacokinetic study of concurrent administration of trastuzumab and high-dose chemotherapy in advanced HER2+ breast cancer. Clin Cancer Res, 2004; 10(21): 7136-7143.
Offring a, R, Vierboom, M P, van der Burg, S H, Erdile, L, and Melief, C J p53: a potential target antigen for immunotherapy of cancer. Ann NY Acad Sci, 2000; 910(223-233; discussion 233-226.
Reilly, R T, Gottlieb, M B, Ercolini, A M, et al. HER-2/neu is a tumor rejection target in tolerized HER-2/neu transgenic mice. Cancer Res, 2000; 60(13): 3569-3576.
Reilly, R T, Machiels, J P, Emens, L A, et al. The collaboration of both humoral and cellular HER-2/neu-targeted immune responses is required for the complete eradication of HER-2/neu-expressing tumors. Cancer Res, 2001; 61(3): 880-883.
Revillion, F, Bonneterre, J, and Peyrat, J P ERBB2 oncogene in human breast cancer and its clinical significance. Eur J Cancer, 1998; 34(6): 791-808.
Rosenberg, S A A new era for cancer immunotherapy based on the genes that encode cancer antigens. Immunity, 1999; 10(3): 281-287.
Ross, J S and Fletcher, J A The HER-2/neu oncogene in breast cancer: prognostic factor, predictive factor, and target for therapy. Stem Cells, 1998; 16(6): 413-428.
Schijns, V E Induction and direction of immune responses by vaccine adjuvants. Crit. Rev Immunol, 2001; 21(1-3): 75-85.
Sharma, S, Karakousis, C P, Takita, H, Shin, K, and Brooks, S P Cytokines and chemokines are expressed at different levels in small and large murine colon-26 tumors following intratumoral injections of CpG ODN. Neoplasia, 2004; 6(5): 523-528.
Slamon, D J, Clark, G M, Wong, S G, Levin, W J, Ullrich, A, and McGuire, W L Human breast cancer: correlation of relapse and survival with amplification of the HER-2/neu oncogene. Science, 1987; 235(4785): 177-182.
Sparwasser, T, Vabulas, R M, Villmow, B, Lipford, G B, and Wagner, H Bacterial CpG-DNA activates dendritic cells in vivo: T helper cell-independent cytotoxic T cell responses to soluble proteins. Eur J Immunol, 2000; 30(12): 3591-3597.
Stan, A C, Casares, S, Brumeanu, T D, Klinman, D M, and Bona, C A CpG motifs of DNA vaccines induce the expression of chemokines and MHC class II molecules on myocytes. Eur J Immunol, 2001; 31(1): 301-310.
Steller, M A Human papillomavirus, it's genes em leader and cancer vaccines. Cancer Cell, 2003; 3(1): 7-8.
Sun, S, Zhang, X, Tough, D F, and Sprent, J Type I interferon-mediated stimulation of T cells by CpG DNA. J Exp Med, 1998; 188(12): 2335-2342.
Takeda, K and Akira, S Toll receptors and pathogen resistance. Cell Microbiol, 2003; 5(3): 143-153.
Takeshita, S, Takeshita, F, Haddad, D E, Ishii, K J, and Klinman, D M CpG oligodeoxynucleotides induce murine macrophages to up-regulate chemokine mRNA expression. Cell Immunol, 2000; 206(2): 101-106.
Takeuchi, O and Akira, S MyD88 as a bottle neck in Toll/IL-1 signaling. Curr Top Microbiol Immunol, 2002; 270(155-167.
Taraban, V Y, Rowley, T F, O'Brien, L, et al. Expression and costimulatory effects of the TNF receptor superfamily members CD134 (OX40) and CD137 (4-1BB), and their role in the generation of anti-tumor immune responses. Eur J Immunol, 2002; 32(12): 3617-3627.
Treurniet, H F, Rookus, M A, Peterse, H L, Hart, M, and van Leeuwen, F E Differences in breast cancer risk factors to neu (c-erbB-2) protein overexpression of the breast tumor. Cancer Res, 1992; 52(8): 2344-2345.
Ueno, T, Toi, M, Saji, H, et al. Significance of macrophage chemoattractant protein-1 in macrophage recruitment, angiogenesis, and survival in human breast cancer. Clin Cancer Res, 2000; 6(8): 3282-3289.
Valmori, D, Levy, F, Miconnet, I, et al. Induction of potent antitumor CTL responses by recombinant vaccinia encoding a melan-A peptide analogue. J Immunol, 2000; 164(2): 1125-1131.
Van den Eynde, B J and Boon, T Tumor antigens recognized by T lymphocytes. Int J Clin Lab Res, 1997; 27(2): 81-86.
van der Bruggen, P, Bastin, J, Gajewski, T, et al. A peptide encoded by human gene MAGE-3 and presented by HLA-A2 induces cytolytic T lymphocytes that recognize tumor cells expressing MAGE-3. Eur J Immunol, 1994; 24(12): 3038-3043.
van der Bruggen, P, Traversari, C, Chomez, P, et al. A gene encoding an antigen recognized by cytolytic T lymphocytes on a human melanoma. Science, 1991; 254(5038): 1643-1647.
Vitiello, A, Marchesini, D, Furze, J, Sherman, L A, and Chesnut, R W Analysis of the HLA-restricted influenza-specific cytotoxic T lymphocyte response in transgenic mice carrying a chimeric human-mouse class I major histocompatibility complex. J Exp Med, 1991; 173(4): 1007-1015.
Vollmer, J, Weeratna, R, Payette, P, et al. Characterization of three CpG oligodeoxynucleotide classes with distinct immunostimulatory activities. Eur J Immunol, 2004; 34(1): 251-262.
Weinberg, A D OX40: targeted immunotherapy—implications for tempering autoimmunity and enhancing vaccines. Trends Immunol, 2002; 23(2): 102-109.
Yamamoto, S, Yamamoto, T, Shimada, S, et al. DNA from bacteria, but not from vertebrates, induces interferons, activates natural killer cells and inhibits tumor growth. Microbiol. Immunol, 1992; 36(9): 983-997.
Yamamoto, T, Kimura, T, Ueta, E, Tatemoto, Y, and Osaki, T Characteristic cytokine generation patterns in cancer cells and infiltrating lymphocytes in oral squamous cell carcinomas and the influence of chemoradiation combined with immunotherapy on these patterns. Oncology, 2003; 64(4): 407-415.
Yu, D, Liu, B, Jing, T, et al. Overexpression of both p185c-erbB2 and p170mdr-1 renders breast cancer cells highly resistant to taxol. Oncogene, 1998; 16(16): 2087-2094.
Zhai, Y, Yang, J C, Spiess, P, et al. Cloning and characterization of the genes encoding the murine homologues of the human melanoma antigens MART1 and gp100. J Immunother, 1997; 20(1): 15-25.

What is claimed is:

1. A conjugate, comprising:
at least one Toll-like receptor (TLR) ligand;
a targeting molecule, wherein the targeting molecule is a peptide or protein that is a ligand for a receptor present on a surface of a desired cell, and wherein the targeting molecule functions to target the conjugate to the desired cell; and
wherein the at least one TLR ligand and the targeting molecule are conjugated together via a cleavable linkage.

2. The conjugate of claim 1, wherein the at least one TLR ligand comprises at least one oligonucleotide containing at least one unmethylated CpG dinucleotide.

3. The conjugate of claim 1 wherein the targeting molecule comprises an immunologically active portion of an immunoglobulin heavy chain.

4. The conjugate of claim 1 wherein the cleavable linkage is a cleavable disulfide linkage.

5. A method of inducing a targeted inflammatory response, comprising the steps of:
providing a conjugate comprising:
at least one Toll-like receptor (TLR) ligand;
a targeting molecule, wherein the targeting molecule is a peptide or protein that is a ligand for a receptor present on a surface of a desired cell, and
wherein the targeting molecule functions to target the conjugate to the desired cell; and
wherein the at least one TLR ligand and the targeting molecule are conjugated together via a cleavable linkage;
administering an effective amount of the conjugate to a subject to induce an inflammatory response at a targeted location.

6. The method of claim 5 wherein, in the step of providing a conjugate, the at least one TLR ligand comprises at least one oligonucleotide containing at least one unmethylated CpG dinucleotide.

7. The method of claim 5 wherein, in the step of providing a conjugate, the targeting molecule comprises an immunologically active portion of an immunoglobulin heavy chain.

8. The method of claim 5 wherein, in the step of providing a conjugate, the cleavable linkage is a cleavable disulfide linkage.

9. The method of claim 5, wherein the targeted location is a tumor.
